(19) United States
(12) Patent Application Publication
(10) Pub. No.: US 2025/0018085 A1
(43) Pub. Date: Jan. 16, 2025

(54) COMPOSITIONS AND METHODS FOR TISSUE REPAIR

(71) Applicants: Washington University, St. Louis, MO (US); The Trustees of Columbia University in The City of The New York, New York, NY (US); Acatechol, Inc., Santa Barbara, CA (US)

(72) Inventors: Guy GENIN, St. Louis, MO (US); Stavros THOMOPOULOS, New York, NY (US); Fei FANG, New York, NY (US); Kollbe AHN, Pasadena, CA (US); Roscoe LINSTADT, Pasadena, CA (US)

(21) Appl. No.: 18/712,368

(22) PCT Filed: Dec. 8, 2022
(86) PCT No.: PCT/US2022/081151

§ 371 Date: May 22, 2024

Related U.S. Application Data
(60) Provisional application No. 63/287,365, filed on Dec. 8, 2021.

(51) Int. Cl. A61L 24/08

(52) U.S. Cl. CPC A61L 24/08; A61L 2430/02; A61L 2430/10

(57) ABSTRACT

Among the various aspects of the present disclosure is the provision of compositions and methods for tissue defect repairs. In some embodiments, the biocompatible adhesive, biocompatible film, or biocompatible material comprises an oxidized chitosan or crosslinked chitosan. In some embodiments, the oxidizing agent or crosslinking agent has been removed to maintain biocompatibility.

### CROSS-REFERENCE TO RELATED APPLICATIONS

This application claims priority from U.S. Provisional Application Ser. No. 63/287,365 filed on 8 Dec. 2021, which is incorporated herein by reference in its entirety.

### STATEMENT REGARDING FEDERALLY SPONSORED RESEARCH OR DEVELOPMENT

This invention was made with government support under AR076008 awarded by the National Institutes of Health and under CMMI1548571 awarded by the National Science Foundation. The government has certain rights in the invention.

### FIELD

The present disclosure generally relates to compositions and methods for repairing tissue defects.

### BACKGROUND

Surgical repairs typically require sutures. Sutured repair and sutured surgical closure can fail when sutures tear through soft tissue, break, constrict blood flow, or otherwise fail to support the healing process. This technology addresses the critical need for adhesive materials to strengthen sutured repairs.

Surgical procedures often require repair of leaks to the dura or the vasculature for hemostasis. This technology additionally addresses the need for materials that can be used as an adhesive or adhesive patch on the dura or to restore hemostasis.

Dental and orthodontic procedures often require bonding of different or similar materials. This technology also addresses the need for effective, biocompatible adhesives for these purposes.

An example is healing and repair of tears to the tendons and entheses of the rotator cuff. Rotator cuff repairs are currently performed arthroscopically using suture anchor systems. Although there are some variations in suturing technique (e.g., single-row vs. double-row) and suture/anchor materials, the repair methods generally rely on transfer of load across only a few anchor and suture grasping points. Mechanical fixation using this approach has reached its limit: increased numbers of strands and anchors do not lead to improved outcomes (i.e., studies have shown that single-row repairs are equivalent to double-row repairs). Therefore, conventional augmentation approaches have focused on enhancing the biology of healing. “Orthobiologics” such as extracellular matrix (ECM) patches, platelet-rich plasma (PRP), and bone marrow-derived stromal cells (MSCs) have been used in an effort to stimulate healing. Patches are typically applied over the repaired tendon to promote healing, e.g., PRP and bone marrow-derived stromal cells stem cell injections are typically injected into the tendon or surrounding space to promote healing. However, there are no rigorous clinical studies showing the efficacy of any of these approaches, and in some cases, trials have been cut short due to negative outcomes. Effective healing of tendon to bone requires the re-integration of the two tissues. To achieve this, there must be good contact between the two tissues (requiring sufficient mechanical fixation) and extracellular matrix must be produced to bridge the two tissues. The current orthobiologic approaches fail to achieve this due to: (1) a lack of mechanical augmentation and (2) poor delivery methods. Generally, the patches, which are placed over the tendon, not between the tendon and bone, are in the wrong place to promote tendon-to-bone healing. Injection of PRP or MSCs is also an ineffective delivery method, as these factors are cleared within a day or two.

There are several emerging solutions to improve rotator cuff repair, primarily driven by large medical device companies and a small number of start-up companies. Mechanically, sutures have been developed with flat and relatively wide cross-sectional areas (compared to the circular cross-sectional area geometry of typical sutures). The flat sutures are an attempt to address the problem of sutures slicing through tendon at grasping points where stress is concentrated. As described above, orthobiologics are also a major area of emerging solutions for rotator cuff repairs. While there is little risk involved with this treatment because the PRP is derived from the patient's own blood, efficacy for rotator cuff repair has never been demonstrated. Similarly, ECM patches have not been effective in any rigorous clinical study.

A few tissue adhesives, for example, fibrin sealant and tissue adhesive, have been on the market for wound closure. However, those adhesives are not designed for repairing tendon-to-bone, because they are not strong enough for holding a tissue-tissue interface together or efficiently transferring load during daily activity after repair and finally lead to higher rates of surgical failure.

Previous work using HP-Chitosan requires several hours of curing time with crosslinking agents at the applied interfaces or surfaces. A recent paper described gelation of 4-arm-polyethylene glycol (PEG)-coupled with catechol-functionalized chitosan via tyrosinase-inspired oxidation with surface-immobilized hematin and hydrogen peroxide (Byun et al, Catalyst-mediated yet catalyst-free hydrogels formed by interfacial chemical activation. Chemical Communications 2014, 50 (22), 2869-2872). This shows great potential for a residual reagent-free gelation of HP-Chitosan, but its main backbone in the literature is PEG, which is not biodegradable. In WO2013077476, HP-Chitosan crosslinked with a thiol group coupling agent has been reported for hemostatic application with its excellent biocompatibility and adhesion and cell-regeneration properties. However, this adhesive hydrogel contains residual reagent, i.e., pluronic (comprising a thiol-containing crosslinking agent). It is presently believed that no literature has shown residual reagent-free HP-Chitosan-Gel as a final product or ingredient to a final product.

### SUMMARY

Among the various aspects of the present disclosure is the provision of compositions and methods for tissue defect repairs.

An aspect of the present disclosure provides for a biocompatible adhesive for use in a subject having a tissue defect, wherein the biocompatible adhesive comprises an oxidized or crosslinked chitosan (e.g., HP-chitosan). In some embodiments, the oxidized or crosslinked chitosan does not comprise an oxidizing agent or crosslinking agent.

Another aspect of the present disclosure provides for a biocompatible film comprising the biocompatible adhesive for use in a subject having a tissue defect, wherein the biocompatible adhesive comprises an oxidized chitosan or crosslinked chitosan.

Yet another aspect of the present disclosure provides for a biocompatible material comprising the biocompatible adhesive and/or the biocompatible film for use in a subject having a tissue defect, wherein the biocompatible adhesive or the biocompatible film comprises an oxidized or crosslinked chitosan.

Yet another aspect of the present disclosure provides for a method of repairing a tissue defect at a tissue defect site comprising: (i) providing the biocompatible film; (ii) contacting the biocompatible film with the biocompatible adhesive of; (iii) applying the biocompatible film to a tissue defect (e.g., bone); and (iv) applying the biocompatible adhesive to the biocompatible film or a tissue defect (e.g., tendon).

Yet another aspect of the present disclosure provides for a method of repairing a tissue defect at a tissue defect site comprising: (i) applying a primer to one side of the tissue defect (e.g., bone) or the other side of tissue defect (e.g., tendon); and (ii) applying the biocompatible adhesive of claim 1 and/or the biocompatible film of claim 2 to the primed tissue defect (e.g., bone) or the other side of tissue defect (e.g., tendon). In some embodiments, the primer is amino-bisphosphonate or a mixture of amino-phosphonates.

Yet another aspect of the present disclosure provides for a method of generating a biocompatible film, biocompatible adhesive, biocompatible material comprising removing an oxidizer in an oxidized chitosan, resulting in a substantially oxidizer-free oxidized chitosan biocompatible adhesive; or optionally drying the biocompatible adhesive resulting in a biocompatible film or applying the biocompatible adhesive to the biocompatible film. In some embodiments, the oxidizing agent or the crosslinking agent has been removed by filtration. In some embodiments, the chitosan is HP-chitosan and is made by reacting primary amine groups of chitosan with carboxylic acid groups of dihydroxy or trihydroxybenzene-(CH₂)n-COOH, n=0-10. In some embodiments, gelation of the HP-chitosan is achieved using a filterable oxidizing crosslinking agent. In some embodiments, the filterable oxidizing crosslinking agent is a periodate form of ionic exchange resins. In some embodiments, the biocompatible adhesive is filtered to remove and/or recycle the oxidizing agent, wherein the oxidizing agent is immobilized onto resin beads. In some embodiments, the oxidizing agent is immobilized onto a porous material, foam, fibrous material, or coated porous, foam, or fibrous material. In some embodiments, a periodate-modified ion exchange resin-bead filtration system is used to oxidize catechol moieties. In some embodiments, the catechol moieties are oxidized to quinones. In some embodiments, the filtration filters off an activating agent and/or resin. In some embodiments, the biocompatible film, biocompatible adhesive, or biocompatible material comprises di- or tri-hydroxy phenyl-functionalized chitosan (HP-chitosan). In some embodiments, the biocompatible film, biocompatible adhesive, or biocompatible material has a shear modulus value between about 1 MPa and about 5 MPa. In some embodiments, the biocompatible film, biocompatible adhesive, or biocompatible material has a shear modulus value of at least about 2 MPa. In some embodiments, the biocompatible film, biocompatible adhesive, or biocompatible material has a shear stress value between about 0.1 MPa and about 0.45 MPa. In some embodiments, the biocompatible film, biocompatible adhesive, or biocompatible material has a shear stress value of at least about 0.35 MPa. In some embodiments, the biocompatible film comprises a rigid, dried HP-Chitosan sheet. In some embodiments, the biocompatible material is a gradient adhesive film comprising a rigid, dried HP-Chitosan sheet, placed onto a bone surface and a viscous filtered HP-Chitosan placed onto a tendon surface. In some embodiments, the biocompatible film and biocompatible adhesive are applied between a tendon and a bone. In some embodiments, the biocompatible film is placed onto a bone surface and the biocompatible adhesive is placed on a tendon surface. In some embodiments, the biocompatible adhesive comprises viscous HP-Chitosan. In some embodiments, the biocompatible adhesive does not comprise an amount of oxidizer (e.g., sodium periodate) sufficient to result in cytotoxicity. In some embodiments, the biocompatible adhesive does not contain a substantive amount of oxidizer (e.g., sodium periodate). In some embodiments, the oxidizer (e.g., sodium periodate) is substantially absent. In some embodiments, the oxidizer (e.g., sodium periodate) is immobilized onto anion exchange resin beads. In some embodiments, anion exchange resin beads are contacted with HP-chitosan and filtered through a syringe filter. In some embodiments, the tissue defect is a torn rotator cuff. In some embodiments, the chitosan is oxidized (e.g., HP-chitosan colloid) and is filtered through periodate-resin beads in a syringe connected to a filter (e.g., a 0.2 μm filter) and evenly spread over a tendon surface resulting in a biocompatible adhesive coated tendon; a biocompatible film (e.g., a dried HP-Chitosan sheet) is applied onto the bone surface resulting in a biocompatible film covered bone; and/or the biocompatible adhesive coated tendon and the biocompatible film covered bone are contacted together. In some embodiments, a tissue defect treated with the biocompatible adhesive, biocompatible film, or biocompatible material of any one of the preceding claims, promotes tenogenesis, shown by greater deposition of Col I and expression of Scx. In some embodiments, the biocompatible adhesive, biocompatible film, or biocompatible material improves the strength of a repair compared to strength of the repair with no biocompatible adhesive, biocompatible film, or biocompatible material. In some embodiments, the biocompatible adhesive, biocompatible film, or biocompatible material promotes long-term tendon-to-bone healing. In some embodiments, the biocompatible adhesive, biocompatible film, or biocompatible material at the tissue defect site increases post-surgical strength of the tissue defect; increases strength of the tissue defect; reduces post-surgical failure of the tissue defect; improves load transfer of the tissue defect; shifts the load from a few anchor points to shear along the biocompatible film, minimizing stress concentration at the tissue defect site; or has at least about a 20% increase in strength compared to the tissue defect site with no biocompatible film used. In some embodiments, the tissue defect site comprises a suture and the suture and the biocompatible film have an increased strength compared to the tissue defect site with no biocompatible film used. In some embodiments, (i) a musculoskeletal injury, a connective tissue-to-bone defect, a connective tissue-to-connective tissue defect, a ligament-to-bone tissue defect, or a tendon-to-bone tissue defect; (ii) a ligament/tendon-to-bone insertion, an articular cartilage-to-bone junction, a hip labrum, an intervertebral disc, a nucleus pulposus-annulus fibrosus-endplates, a cementumperiodontal ligament-alveolar bone, a muscle-to-tendon, an inhomogeneous or anisotropic tissue, a knee meniscus, a temporomandibular joint disc, a root-periodontium complex, a tendon-bone insertion, a synovial joint, or a fibrocartilaginous tissue; or (iii) a flexor tendon, a rotator cuff (optionally, a tear to the tendon or enthesis of the rotator cuff), an anterior cruciate ligament, a meniscus, or an Achilles tendon.

Yet another aspect of the present disclosure provides for a use of biocompatible film, biocompatible adhesive, biocompatible material, or method of any one of the preceding claims for general surgery as a hemostatic and/or adhesive agent.

Yet another aspect of the present disclosure provides for a use of the biocompatible film, biocompatible adhesive, biocompatible material, or method of any one of the preceding claims as an osteoinductive binder or agglomerator.

Yet another aspect of the present disclosure provides for a use of the biocompatible film, biocompatible adhesive, biocompatible material, or method of any one of the preceding claims for endodontic applications.

Yet another aspect of the present disclosure provides for a use of the biocompatible film, biocompatible adhesive, biocompatible material, or method of any one of the preceding claims wherein the biocompatible film, biocompatible adhesive, biocompatible material, or method of any one of the preceding claims as a coating to a suture that is used in a repair.

Yet another aspect of the present disclosure provides for a use of the biocompatible film, biocompatible adhesive, biocompatible material, or method of any one of the preceding claims for orthodontic applications and dental implants.

Yet another aspect of the present disclosure provides for a use of the biocompatible film, biocompatible adhesive, biocompatible material, or method of any one of the preceding claims for repair of the dura, including leakages.

Yet another aspect of the present disclosure provides for a use of the biocompatible film, biocompatible adhesive, biocompatible material, or method of any one of the preceding claims for repair of vasculature.

Yet another aspect of the present disclosure provides for a use of the biocompatible film, biocompatible adhesive, biocompatible material, or method of any one of the preceding claims for dental or orthodontic procedures which often require bonding of different or similar materials.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is an exemplary embodiment of a schematic of bioadhesive synthesis in accordance with the present disclosure. Chitosan was 1) functionalized with catechol, 2) filtered through periodate ionic resin beads, and 3) used to adhere tendon to bone.

FIG. 2(A-D) is an exemplary embodiment of mechanical properties of the BGC bioadhesive in accordance with the present disclosure. FIG. 2A shows that to ensure firm contact during adhesive curing, tendon and bone were clamped overnight. FIG. 2B shows lap shear experiments were performed on adhered tendon and bone planks. FIG. 2A shows how the BGC adhesive was placed between tendon and bone. FIG. 2C shows failure surfaces after completion of shear lag tests are shown for fibrin, BGC bioadhesive, and CA. FIG. 2D shows representative force-elongation curves, shear force, shear stress, shear modulus, toughness, and resilience of adhered bone-tendon planks for the three adhesives. The two dashed lines in each plot show the range of mechanical parameters of adhesives published in the literature. *p<0.05; **p<0.001; ***p<0.0001; ****p<0.00001 based on ANOVA followed by Tukey's post hoc tests. DP-chit., DP-chitosan. All data are presented as mean±standard deviation.

FIG. 3 is an exemplary embodiment of in vitro analysis of adhesive biocompatibility in accordance with the present disclosure.

FIG. 4(A-E) show exemplary embodiments of cell density and viability analyses of after in vitro culture of tendon fibroblasts with adhesives (analyses were conducted after 1 d (Day1), 3 d (Day 3), and 5 d (Day5) of culture), as well as proliferation analysis after in vitro co-culturing of tendon cells with fibrin, bioadhesive, bioadhesive created without the use of the bead filter system (Unrefined bioadhesive), and Loctite cyanoacrylate (CA), in accordance with the present disclosure. FIG. 4A shows representative phase contrast microscope images of tendon cells for D1 (top row) and representative fluorescent images of tendon cells co-cultured with four adhesives at D1, D3, and D5 (rows 2, 3, and 4, respectively). FIG. 4B shows cell density per 0.4 mm2 after culturing for D1, D3, and D5. FIG. 4C shows analysis of cell death, represented as the percentage of dead cells relative to all cells at D1, D3, and D5. Red staining, dead cells; Green staining, live cells. p<0.05 based on ANOVA followed by Tukey's post hoc tests is indicated by bars that do not share a common letter. All data are presented as mean±standard deviation. FIG. 4D is a representative histogram showing that cells are in different proliferation stages, manually subdivided into proliferating stage 1 (P1), 2 (P2), 3 (P3), and 4 (P4). FIG. 4E shows percentage of cells at each of the four proliferation stages, normalized by the total number of cells after co-culturing cells with four adhesives for 5 days. P<0.05 between groups is indicated by the letters “a”, “b”, “c”.

FIG. 5 is an exemplary embodiment of gene expression in accordance with the present disclosure. Gene expression of tenogenic markers after in vitro culturing of tendon cells with fibrin, BGC, unrefined BGC, and CA for 1 d. p<0.05 based on ANOVA followed by Tukey's post hoc tests is indicated by bars that do not share a common letter. All data are presented as mean±standard deviation.

FIG. 6(A-C) is an exemplary embodiment of immunofluorescence analysis for protein expression of collagen type I and Scx after in vitro culturing of tendon cells with fibrin, BGC, unrefined BGC, and CA for 1 d, in accordance with the present disclosure. FIG. 6A shows representative images of fluorescent staining of collagen type I (Col I) and Scx. The second and fourth rows show magnified images of the corresponding white boxes in the first and third rows, respectively. Red, collagen type I or Scx; blue, nuclei. FIG. 6B shows intensity of collagen type I staining for tendon cells co-cultured with each of the four adhesives. FIG. 6C shows percentage of Scx-positive (Scx+) tendon cells cultured with each of the four adhesives. p<0.05 based on ANOVA followed by Tukey's post hoc tests is indicated by bars that do not share a common letter. All data are presented as mean±standard deviation.

FIG. 7 is an exemplary embodiment of shear modulus and shear stress in accordance with the present disclosure. Shear modulus (left) and peak shear stress (right) at failure for an adhesive repair between tendon and bone ex vivo achieved with the disclosed chitosan-HP adhesive and Primer3 led to improved mechanical properties of the repair.

### DETAILED DESCRIPTION

As described herein, “HP-Chitosan” can include dihydroxy benzyl/phenyl- and trihydroxy benzyl/phenyl-conjugated chitosan. HP-Chitosan is chitosan functionalized with 3,4,5-trihydroxy, 2,3-, 3,4-, or 4,5-dihydroxy benzyl or phenyl moiety. Here, inventors developed a method of preparing an HP-Chitosan gel that does not contain any residual reagents or residual crosslinkers in the final product formula via oxygen-involved incubation and/or by consuming all the crosslinkers during the production before packaging, and/or the method to remove all the catalysis and un-reacted crosslinkers during the production process and/or during preparation in clinical practice. This gel (pre-crosslinked) without containing any crosslinkers as ingredient can be applied for adhesives, cosmetics, as anti-microbial, anti-acne-, and anti-aging agents as catechol is a radical scavenger and anti-oxidant found in plant leaves and fruits.

The technology can include methods and uses of HP-Chitosan-Gel with a viscosity range of 10,000-200,000 cps, and presents alternatives to overcome the limitations posed by previous studies. One example of an application is maxillofacial surgery. In maxillofacial surgery, titanium meshes, expanded polytetrafluoroethylene, collagen membranes, polylactic acid membranes, and/or collagen-based and hyaluronic acid-based binders are conventionally used. HP-Chitosan has shown adequate tensile strength in a wet/saliva/blood environment. Review of prior art shows that there is no literature describing the use of HP-Chitosan-Gel with shear-thinning properties as an orthopedic surgery aid to assist or replace sutures, biodegradable osteoinductive binders, antimicrobial agents, surgical glues, ingredients of cosmetics, or feminine products.

### Preparation of HP-Chitosan Solution/Colloid

Chitosan (1 g, 6.5 mmol for the monomer stoichiometry of M.W. 100 kDa, 70% deacetylated) was dissolved in a HCl solution (100 mL, pH 2), and the pH was adjusted to 5 using 0.1M HCl. 1-ethyl-3-(3-dimethylamino-propyl)-carbodiimide hydrochloride (EDC, 1.24 g, 6.5 mmol) and 3,4-dihydroxy hydrocinnamic acid (HCA, 1.18 g, 6.5 mmol) were dissolved in a co-solvent of double distilled water (DDW) and ethanol (1:1 v/v), then added to the chitosan solution. The reaction time was 12 hrs, and the pH was maintained between 4.5 and 5 during the entire reaction time. The final product was dialyzed by a membrane (MWCO: 12,000-14,000 Da) against 10 mM NaCl solutions (pH<5) for 2 days, then further dialyzed followed by DDW for 4 hrs. The degree of catechol functionalization was 12.4%, as determined by ¹H-NMR.

### Preparation of Periodate-Form of Ionic Exchange Resin Beads as Catechol Oxidizing Agent

Periodate is an excellent oxidizing agent to catecholic moieties to quinones. However, it is strongly oxidizing and is thus cytotoxic to cells. In the present disclosure, a periodate form of ionic exchange resins was prepared by modifying commercially available ionic exchange resins (e.g., Amberlyst A26, Amberlite IRA 904). The periodate resins were added to a syringe equipped with a filter and the HP-Chitosan was pushed through the resin and filter. Gelation of the HP-Chitosan was initiated when the HP-Chitosan colloid passed through the resin and filter and exited the syringe, curing over a period between 0.01-48 hours. The volume ratio of HP-Chitosan colloid to oxidizing agent-immobilized resins will vary based on the size of the chitosan, concentration, pH, and type of resin. Importantly, no resin remained in the HP-Chitosan after the HP-Chitosan passed through the filter.

### Preparation of Hematin-Immobilized Polystyrene Resin Beads as Catechol Oxidizing Agent

Hematin is reported as an alternative catalyst to horseradish peroxidase for in situ gelation of polymers with phenolic hydroxyl moieties. Gelation in an aqueous media of gelatin and chitosan derivative with phenolic hydroxyl groups moieties has been reported in the presence of hematin and H₂O₂. However, hematin binds and disrupts cell membranes causing the cytotoxicity of cells at a high concentration when the gelation occurs in situ. In the present disclosure, hematin is immobilized on amine-functionalized (or amine-activated) resins or beads (such as, not limited to, amine-functionalized polystyrene or agarose resin) via carbodiimide peptide coupling chemistry. An illustrative example is given as follows: hematin porcine (4.1 mg, 0.0065 mmol) is dissolved in a co-solvent of DDW and DMF (1:1 v/v), and then 1 M NaOH is added to adjust the pH to 12. After reducing the solution pH to 8.5, EDC (5 mg, 0.026 mmol) and N-Hydroxy Succinimide (NHS, 0.75 mg, 0.0065 mmol) are added to the hematin solution. The solution is then added to the amine-functionalized resin. The surface change of the resin from primary to hematin is monitored and confirmed by FTIR and XPS.

### Application of HP-Chitosan Adhesives

The present disclosure is based, at least in part, on the discovery that a catechol-derived adhesive can increase the strength of a tendon-to-bone repair to 6-9-fold of commercially available tissue adhesives and prompted improved cellular responses in vitro compared to off-the-shelf adhesives. As shown herein, the new adhesive has nearly an order-of-magnitude higher shear strength than other available adhesives, making it appropriate for high load medical applications (e.g., tendon-to-bone repair).

Conventional suture-based surgical techniques used to repair torn rotator cuff tendons do not result in a mechanically competent tendon-to-bone attachment, leading to high failure rates post-operatively. These repairs fail because all of the muscle load is concentrated on a few finite suture anchor points, in sharp contrast to the even distribution of loads across a wide footprint in the healthy attachment. The presently disclosed adhesive and materials are the first to meet the clinical needs: high mechanical strength, low modulus, no cytotoxicity, an ability cure on wet surfaces, and an ability to promote healing responses.

The presently disclosed design of bio-inspired catechol-derived adhesive can achieve the appropriate or sufficient mechanical strength required for rotator cuff repair (current surgical techniques fail at 200-400 N) and help load transfer across a relatively larger repaired region to avoid surgical failure. As such, the described compositions and methods do not fail at about 200 to 400 N or more.

Additionally, the experiments disclosed herein have demonstrated that the adhesive material is as biocompatible as commercially available adhesives and also promotes tenogenesis in vitro. All these data indicate that the presently disclosed design will serve as a better solution for rotator cuff injury/degeneration to support loading and potentially achieve regeneration.

The biomechanical and biological features of this adhesive can be expanded to other orthopedic tissues (i.e., cartilage, ligament, meniscus, intervertebral disc), wound closure, and for a variety of other tissue applications.

### Adhesive Material for Tendon-to-Bone Repair

Rotator cuff repair to recover shoulder function is one of the most common orthopedic surgical procedures, with over 600,000 repairs performed each year in the United States. It is the most common shoulder condition, with more than 17 million individuals in the United States affected. Recreating a mechanically competent attachment between the ruptured tendon and bone is necessary to successfully transfer muscle load from tendon to bone and achieve shoulder function.

A range of mechanisms exists in the healthy tendon-to-bone attachment (the enthesis) to distribute force over a relatively large attachment footprint and thereby reduce stress concentrations that would otherwise at the interface between two tissues with dramatically different mechanical properties (i.e., tendon and bone). Unfortunately, this attachment system is not recreated with current suture-based surgical techniques and is not regenerated during healing, leading to high failure rates post-operatively (e.g., re-rupture rates after rotator cuff repair are 20-94%, depending on the patient population). The remarkable failure rates after surgical repair are in large part due to the repair techniques used to secure tendon to bone: instead of distributing the load across a wide attachment footprint area, as in the healthy attachment, surgical repairs concentrate stress on a small number of suture anchor points. These stress concentrations lead sutures to tear through the tendon, motivating the development of technologies that distribute stresses away from suture anchors and across the attachment footprint.

Here, mechanically-optimized adhesive materials have been developed to implant between tissue interfaces for mimicking the natural load transfer mechanism of tendon-to-bone attachment and increasing the load tolerance of the repair.

The report is based on the findings that a mussel-inspired catechol-derived adhesive can increase the strength of a tendon-to-bone repair to 6-9-fold of commercially available tissue adhesives. Additionally, the new adhesive prompted improved cellular responses in vitro compared to off-the-shelf adhesives.

### Materials, Components, and Concentration of Chemical Compounds Used

Chitosan has been traditionally used as a hemostatic agent and a wound dressing due to its unique ability to attach to human tissues and blood cells. Chitosans have additionally shown potential to improve osteoconductivity and natural antibiocide/antimicrobial. Inspired by marine mussel's catechol chemistry, di- or tri-hydroxy phenyl-functionalized chitosan (termed “HP-Chitosan”) has demonstrated potential for biomedical applications with its improved solubility in neutral or less acidic aqueous media. However, despite the uses of toxic oxidizing agents such as sodium periodate or the addition of crosslinkers, no meaningful shear bonding strength for tendon-to-bone repair has been reported in the literature. Moreover, elastic modulus differences of soft-hard tissue interfaces between tendon and bone have presented insurmountable challenges to the design of an effective bioadhesive.

HP-Chitosan adhesive is not yet commercially available, but is used in hundreds of published articles, and is a preferred embodiment of the invention. The preferred concentration of HP-Chitosan is from 1 wt. % to 30 wt. %. A primer used herein includes an amino-bisphosphonate such as alendronic acid, which is commercially available, and represents a preferred embodiment. The preferred concentration of amino-bisphosphonate is saturated. For example, the concentration of alendronic acid can be 1-200 mg/mL alendronic acid in water with sufficient HCl or other strong acid to dissolve (e.g., 10-3000 μM HCl). In an embodiment, 20 mg/ml of alendronic acid solution was made with 280 μM HCl.

Here both (1) the cytotoxicity issue of periodate and (2) the elastic modulus mismatch issue of the soft-hard tissue interface have been addressed. To solve these issues, (1) the strong oxidizing agent was immobilized onto ionic exchange resin beads for filtration to remove the oxidant, and (2) a gradient adhesive scaffold was built by applying a rigid, dried HP-Chitosan sheet onto a bone surface while placing viscous HP-Chitosan on tendon surface.

These adhesive materials can be applied as a biocompatible film, a biocompatible layer, or an injection of liquid adhesive solutions such as hydrogels. When a standard surgical repair is performed, the film, the layer, and/or the hydrogel can be interposed between the tendon and bone. The adhesive can adhere to the tendon on one side and bone on the other side, providing shear resistance and improving the strength of the tendon-to-bone repair. During the healing process, the adhesive material will promote new tissue formation and finally realize, at least in part, regeneration of tendon-to-bone attachment.

The compositions, systems, and methods as described herein can provide support for surgical and tissue defect/injury sites in tissue-tissue interfaces and multi-tissue interfaces, including but not limited to ligament/tendon-to-bone insertion, articular cartilage-to-bone junction, hip labrum, intervertebral disc (nucleus pulposus-annulus fibrosus-endplates), cementum periodontal ligament-alveolar bone, muscle-to-tendon, inhomogeneous or anisotropic tissues such as knee meniscus or temporomandibular joint disc, root-periodontium complex, tendon-bone insertion, synovial joints, or fibrocartilaginous tissues.

More generally, a tissue defect, which can be treated with the compositions and methods as described herein, can be any injury resulting from damage of tissue at a tissue defect site or at the interface of the muscle and skeleton, which can be due to strenuous activity.

According to different application scenarios, mechanical parameters (i.e., shear force, shear stress, shear modulus, toughness) of adhesive materials can be tuned by optimizing the adhesive composition and their relative concentration of oxidizing reagents to match the required strength of applied tissues. These optimization processes can be achieved by conducting shear lag experiments and finite element analysis to evaluate and predict the ability of adhesive interlayers to improve load transfer across a multi-tissue interface. Additionally, adhesive materials can be impregnated with growth factors or other biochemical drugs to enhance new tissue deposition and integration at injured regions with adhesive degradation. The selection of appropriate growth factors or biochemical components can be evaluated by in vitro biocompatibility experiments.

Mechanical tests were conducted finding that disclosed embodiments of the adhesive material achieved significantly increased mechanical strength applied on the tendon-to-bone interface compared to currently available tissue adhesives on the market. A series of in vitro culturing experiments also demonstrated that this developed adhesive was biocompatible, demonstrated by 95% cell viability comparable to FDA-approved tissue adhesives, and had better healing responses, demonstrated by promoted tenogenesis. Optimization of the adhesive strength by tuning adhesive components and regulating crosslinking reagents is underway. Cadaver rotator cuff repair experiments and preclinical animal research are also underway.

HP-Chitosan is toxic if it contains crosslinking or oxidizing agents such as sodium periodate. According to the present disclosure, an oxidizing agent (e.g, periodate) is immobilized on commercially available resin beads through which the adhesive is filtered. The adhesive is then cured via oxidation-mediated crosslinking.

Rotator cuff injury is common: approximately 50% of the population over the age of 65 has a rotator cuff tear. Rotator cuff repair is one of the most common orthopedic surgical procedures, with over 600,000 repairs performed each year, affecting more than 17 million individuals in the United States. Repairs are often too weak to accommodate the high forces transferred across the shoulder, leading to repair-site failures that require reoperation. Clinical studies have shown failure rates ranging from 20-94%. Loss of strength and function results in permanent disability and leads to occupational challenges and recreational limitations for patients. The presently disclosed adhesive material significantly improves the mechanical strength of tendon-to-bone repair through enhanced load distribution across the repair site and healing responses, to reduce the number of cuff repair failures and improve postoperative outcomes.

### Chitosan

Chitosan is a linear polysaccharide composed of randomly distributed β-(1→4)-linked D-glucosamine and N-acetyl-D-glucosamine (acetylated unit). It is commonly made by treating the chitin shells of shrimp and other crustaceans with an alkaline substance such as sodium hydroxide. In the present disclosure, chitosan is the precursor to HP-Chitosan and chitosan is commonly obtained as a mixture of polymers and smaller oligomers of varying lengths. HP-Chitosan, therefore, is also polymeric in nature, and thus includes smaller oligomers with small numbers of repeating glucosamine subunits (n=3-5, or 5-10) as well as longer polymers (n=10-100, 100-1000, 1000-10,000, 10,000-100,000) depending on the source of the chitosan. Since chitosan is commonly prepared by deacetylation of chitin, HP-Chitosan may contain varying amounts of N-acetylated subunits. In some embodiments HP-Chitosan may have 0%, 0.001-0.01%, 0.01-0.1%, 0.1-1%, 1-10%, 10-20%, 20-40%, or 40-50%, N-Acetylation.

The degree of N-Acetylation in polymeric β-(1→4)-linked D-glucosamine species determines whether the polymer is classified as chitosan or as chitin; when there is >50% N-acetylation, the polymer is classified as “chitin”, when there is <50% N-Acetylation, the polymer is classified as “chitosan”, and when there is exactly 50% N-acetylation, the polymer may be classified as either.

Chitosan has been covalently modified to contain ortho-dihydroxy or -trihydroxyl phenyl (or benzyl) moieties in prior arts for biomedical applications. For example, catechol-functionalized chitosan has been used in hemostatic applications. Chitosan has also been modified to contain a 2,3-, 3,4-, or 4,5-dihydroxy or 2,3,4- or 3,4,5-trihydroxy phenyl (or benzyl) moiety. In the present disclosure, the di- and tri-hydroxy phenyl-functionalized chitosan formulations are termed “HP-Chitosan”. Phenyl (or benzyl) rings with ortho-dihydroxy or -trihydroxy functionality do not necessarily need to be directly attached from the chitosan to the phenyl ring to qualify the material as “HP-Chitosan”.

“HP-Chitosan” can include compounds of the formula Y wherein
Y is a polymer of the following structure:

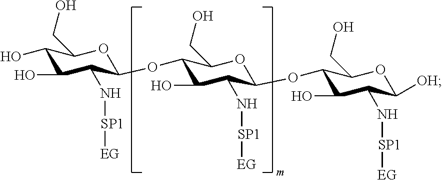

where m is an integer with a value such that 3≤m≤100,000;
and where each SP1 in the entire polymer is independently absent or selected from the group consisting of —(CH₂)₁₋₁₀—, —(C═O)—, —C═O(CH₂)₁₋₁₀—, —(C═O)O—, —(C═O) NH—, —(CH₂CH₂O)₁₋₁₀—, —CH₂CH₂NH—, —(C═O) CH₂CH₂—, —CH₂(CHOH) CH₂O—, —(C═O)CH(NH₂)CH₂—, or —(C═O)CH═CH—;
and where each EG in the entire polymer is independently selected from the group consisting of —H, —CH₃, —(C═O) CH₃, or Z:
where Z is the group consisting of:

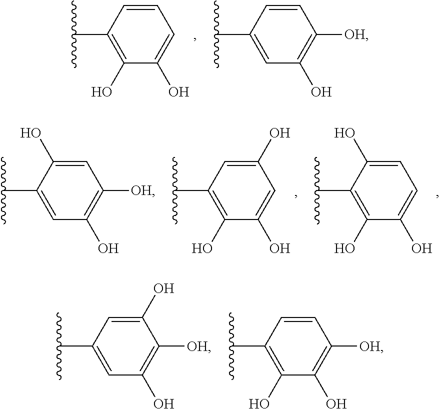

or dopamine;
provided that there is at least one Z group present in the polymer when m=3-10;
and provided that there are at least 2 Z groups present in the polymer when m>10
and provided that for integer values of m>20, there is at least one Z group for each hundred integer values of m.

HP-Chitosan has demonstrated tremendous potential for biomedical applications with its improved solubility in neutral or less acidic aqueous media (Ryu, J. H.; Lee, Y.; Kong, W. H.; Kim, T. G.; Park, T. G.; Lee, H. Catechol-functionalized chitosan/pluronic hydrogels for tissue adhesives and hemostatic materials. Biomacromolecules 2011, 12 (7), 2653-2659). HP-Chitosan shows excellent biocompatibility and biodegradability.

However, it is believed that there has been no prior disclosure of any method to produce flowable gel or hydrogel (with a viscosity range of 10,000-200,000 cps) without incorporating other types of polymer backbones or without the addition of crosslinkers.

For example, a recent paper described gelation of 4-arm-polyethylene glycol (PEG)-coupled with catechol-functionalized chitosan via tyrosinase-inspired oxidation with surface-immobilized hematin and hydrogen peroxide (Byun et al, Catalyst-mediated yet catalyst-free hydrogels formed by interfacial chemical activation. Chemical Communications 2014, 50 (22), 2869-2872). This showed great potential for a residual reagent-free gelation of HP-Chitosan, but its main backbone in the literature was PEG, which is not biodegradable. Furthermore, 4-arm PEG is extremely expensive, and is therefore impractical for wide use.

In WO2013077476, HP-Chitosan itself was a colloid or solution (with a viscosity<10,000 cps). After crosslinking with thiol group-containing coupling agent, HP-Chitosan solution became a gel, and was used for hemostatic application with excellent biocompatibility, adhesion, and cell-regeneration properties. The crosslinker used was pluronic (comprising a thiol-containing crosslinking agent).

It is believed that no literature has shown HP-Chitosan-Gel (residual reagent-free) as a final product or ingredient to a final product.

Described herein are new methods of gelation of HP-Chitosan (HP-Chitosan-Gel) via a one-pot gelation process without any crosslinker and without oxidizing agents such as sodium periodate.

Described herein are new methods of gelation of HP-Chitosan (HP-Chitosan-Gel) via one-pot gelation process with water-soluble amine-crosslinkers such as bis-sulfosuccinimidyl suberate (BS3), succinic acid, glutaric acid, glyoxal, and/or glutaraldehyde, and methods of their uses.

Described herein are new methods of gelation of HP-Chitosan (HP-Chitosan-Gel) through the removal of water and partial oxidative crosslinking via oxygen-involved incubation.

Described herein are new methods of gelation of HP-Chitosan (HP-Chitosan-Gel) using catalyst-immobilized polymer resin beads, and methods of their uses.

Described herein, “HP-Chitosan-Gel” is a hydrogel with a viscosity range of 10,000-200,000 cps. “HP-Chitosan-Gel” can include the polymers of formula Y, which have been subsequently partially crosslinked, and do not contain any residual reagents used to induce crosslinking.

In some embodiments, “HP-Chitosan” also includes polymers Y which have been crosslinked and processed into a gel or hydrogel, such that the resulting gel does not contain any residual crosslinkers or oxidizing agents. In some embodiments, this gel is obtained by consuming all the crosslinkers (pre-polymerization or oligomerization) during the production before packaging. In some embodiments, this technology describes methods to remove all the catalysts and un-reacted crosslinkers during the production process and/or HP-Chitosan gels (HP-Chitosan-Gel) or during its preparation in clinical practice. In some embodiments, this HP-Chitosan-Gel can be applied as an anti-microbial, anti-acne-, anti-oxidant, wound-closure, and/or hemostatic agent in surgery, drugs, and/or cosmetics.

### Development of Amino-Bisphosphonate-Based Bone Surface Primers.

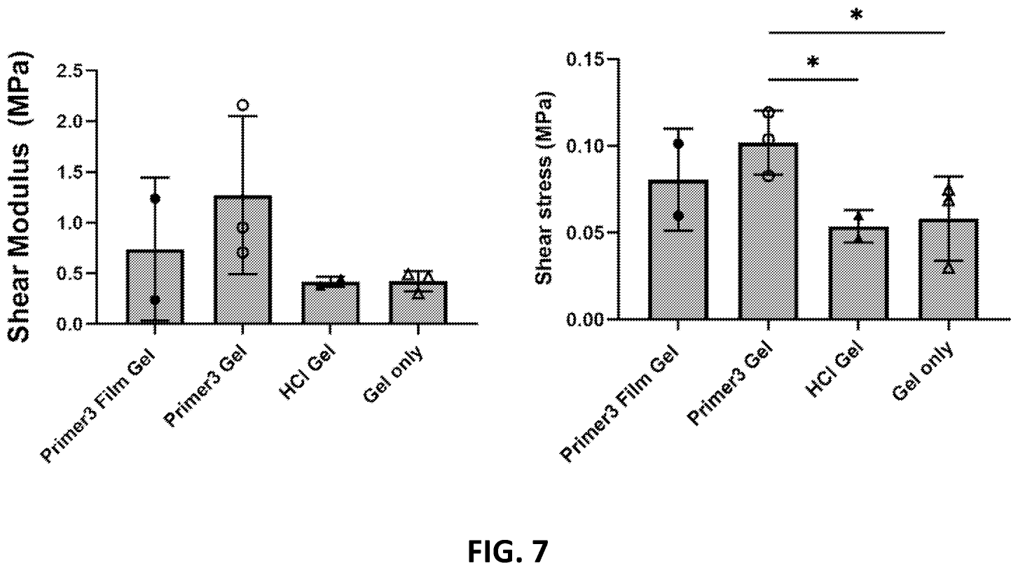

To enhance adhesion to bone, alendronic acid (a small molecule in the amino-bisphosphate class of drugs) was used to prime the bone surface. Amino-bisphosphonates have a high affinity to calcium phosphates, including hydroxyapatite in bone, and are used to reduce bone resorption in treatment of osteoporosis. This high affinity for hydroxyapatite makes alendronic acid a promising primer for further improving the bioadhesive's adhesion to bone. Three primers were tested for their potential to enhance adhesion to bone: phosphate-methacrylate in water, alendronic acid in water/HCl/ethanol at a high concentration, and alendronic acid in water/HCl at a low concentration. The combination of alendronic acid-mediated priming and HP-chitosan adhesive (Primer3 Gel, FIG. 7) doubled the shear stress/strength (0.11 MPa) compared to a previously reported bioadhesive (biocompatible adhesive+film without the bisphosphonate primer) and produced a shear modulus (1.6 MPa) that lands the bioadhesive system in the regime in which it will yield a “repair strength increase” for tendon-to-bone repair. The concentration of the solution was 1-200 mg/ml alendronic acid in water with sufficient HCl or other strong acid to dissolve (e.g., 10-3000 μM HCl). A 20 mg/mL solution was made with 280 μM HCl.

### General Surgery

To overcome its low viscosity or poor water solubility, chitosan was modified chemically to show its potential for hemostatic applications. For example, catechol functional groups have been conjugated to chitosan via carbodiimide peptide coupling chemistry using EDC (1-Ethyl-3-(3-dimethylaminopropyl) carbodiimide).

Although HP-Chitosan has demonstrated great potential for biomedical applications with its improved solubility in neutral or less acidic aqueous media (Ryu et al., Biomacromolecules 2011, 12 (7), 2653-2659), the methods of use of HP-Chitosan in previous work include applying a mixture of HP-Chitosan and crosslinkers directly to interfaces without removal of residual reagents (WO2013/077476).

Described herein are methods of preparing and the uses of HP-Chitosan-Gel (pre-polymerized, residual reagent-free HP-Chitosan hydrogel) for general surgery as hemostatic and/or adhesive materials.

### Bone Repair and Osteoinductive Binder/Agglomerator for Bone Graft Materials/Substitutes and its Methods of Use

A bone graft is a procedure that replaces and regenerates lost bone to create a solid base for implant treatment. Several bone graft substitutes such as allografts, xenografts, and alloplasts are used as a filler. Clinically, xenograft bone substitutes, such as deproteinized bovine bone mineral, are typically used in a hydrated form, mixed with saline or the patient's blood, although these substitute particles are not well agglomerated. Because xenograft bone substitute particles are not well retained at the defect site, their displacement reduces potential osteoconductivity. One approach to solve this clinical problem was to employ non-resorbable materials such as titanium mesh or resorbable materials such as collagen membranes. Despite some success, non-resorbable barrier membranes require follow-up surgery to remove the materials. Other approaches such as collagen meshes or hyaluronic acid do not have the necessary rigidity and osteoinductivity.

Chitosan has traditionally been used as a hemostatic agent and wound dressing due to its unique ability to attach to human tissues and blood cells. Chitosans have additionally shown potential to improve osteoconductivity and are a natural antibiocide/antimicrobial. Phosphorylated chitosan-based membranes have shown higher adsorption of bone morphogenetic protein mimic (cytochrome C) and improved osteoinductivity (Bombaldi de Souza, et al, Phosphorylation of chitosan to improve osteoinduction of chitosan/xanthan-based scaffolds for periosteal tissue engineering. International Journal of Biological Macromolecules 2020, 143, 619-632). Kung et al. reported that chitosan-collagen composites may induce in vivo new bone formation around pure titanium implant surfaces (Kung et al, The osteoinductive effect of chitosan-collagen composites around pure titanium implant surfaces in rats. Journal of Periodontal Research 2011, 46 (1), 126-133). However, due to the poor solubility of chitosan, it has been used only as a membrane material.

HP-Chitosan has not been used as an osteoinductive binder/agglomerator despite its excellent antimicrobial, tissue-regeneration promoting, and hemostatic properties. Described herein is HP-Chitosan-Gel as osteoinductive binder/agglomerator.

### Antimicrobial Gel for Endodontic Applications

More than 15 million root canals are done each year, according to the American Association of Endodontists. During the procedure, the tooth's pulp and nerve are removed before the tooth is cleaned and sealed. If bacteria, viruses or yeasts contaminate the tooth, another root canal procedure or surgery must be done. Chitosan has not been used for this application due to its poor water solubility despite its excellent antimicrobial, tissue-regeneration promoting, and hemostatic properties. Described herein is HP-Chitosan-Gel as endodontic applications such as implant and root canal treatments.

### Adhesive Agent and Adhesive Materials

As described herein are adhesive agents and adhesive materials.

The adhesive agent can comprise a chitosan-based agent, such as an oxidized di- or tri-hydroxy phenyl-functionalized chitosan (e.g., termed “HP-Chitosan”). The oxidizing agent (e.g., periodate) can be immobilized on ionic exchange resin beads and via filtration. As disclosed herein, adhesives and adhesive materials comprising the chitosan-based adhesive agent are free of additives such as crosslinking agents and toxic oxidizing agents via the immobilization and filtration process.

Chitosan is a linear polysaccharide composed of randomly distributed β-linked D-glucosamine and N-acetyl-D-glucosamine. It can be made by treating the chitin shells of shrimp and other crustaceans with an alkaline substance, such as sodium hydroxide.

As an example, an adhesive material can comprise a gradient adhesive scaffold comprising a rigid, dried HP-Chitosan sheet placed onto a bone surface while placing viscous HP-Chitosan on a tendon surface.

As described herein, an adhesive can be implanted in between tissue interfaces. These adhesive agents can be applied as a biocompatible film, a biocompatible layer, or an injection of liquid adhesive solutions such as hydrogels.

The adhesive can mimic the natural load transfer mechanism of tendon-to-bone attachment and increase the load tolerance of the repair.

Biocompatible adhesives have been conventionally used for wound closing and not in tendon/ligament applications. The use has been limited to interface-aligned tissues. Adhesives for use in implanting between tissue interfaces, layers, or films, as described herein can have properties such as biocompatibility, compatible interfacial and bulk strength, compatible modulus, compatible swelling, and a sufficiently long shelf-life. Other adhesives can be used in combination with the adhesive agent as described herein, such as catechol-based adhesives, mechanically-based adhesives, bio-glues (e.g., fibrin-based, albumin-based), ionic polymer adhesives, biodegradable polyester adhesives, polysaccharide-based adhesives, or collagen-binding adhesive.

In some embodiments, an adhesive agent can be tailored to optimize penetration of the adhesive into soft tissue at a specific depth and nature of penetration. An adhesive can superficially adhere a tissue surface to another tissue surface (e.g., soft tissue or bone) or the adhesive can penetrate a tissue surface into the depth of a tissue.

In some embodiments, an adhesive agent can include those defined to penetrate distances ranging from nanometers to millimeters into a fibrous tissue. An optimal range of depth is between one and ten times the mean spacing of fibers.

In some embodiments, the range of depth of adhesive penetration into a soft tissue can be between about 1 nm and about 1 cm.

For example, the adhesive can have a penetration depth of about 1 nm; about 100 nm; about 200 nm; about 300 nm; about 400 nm; about 500 nm; about 600 nm; about 700 nm; about 800 nm; about 900 nm; or about 1000 nm.

As another example, the adhesive can have a penetration depth of about 1 mm; about 100 mm; about 200 mm; about 300 mm; about 400 mm; about 500 mm; about 600 mm; about 700 mm; about 800 mm; about 900 mm; or about 1000 mm.

Recitation of each of these discrete values is understood to include ranges between each value. Recitation of each of a range is understood to include discrete values within the range.

Processes for defining desirable adhesive properties for adhesives to coat sutures are well known; see e.g., Linderman et al. 2015 Acta Biomat. 23 229-239; U.S. Pat. No. 10,631,973 (WU Docket No. 014989-CIP1/1); and U.S. Pat. No. 10,314,574 (WU Docket No. 014989-ORD1/1) are incorporated by reference in their entireties. Except as otherwise noted herein, therefore, the process of the present disclosure can be carried out in accordance with such processes.

### Tissue Defect

As described herein are compositions and methods to provide a mechanically competent attachment between tissues, such as a ruptured tendon and bone.

The compositions and methods described herein can be used to treat a tissue defect. As described herein, a tissue defect can be a tissue-to-tissue defect, for example, a ligament/tendon-to-bone injury, connective tissue to bone, connective tissue to connective tissue, or a musculoskeletal injury. A repair of the tissue defect site can be strengthened with the use of a film as described herein. Repair of tendon/ligament to bone has been a difficult challenge in orthopedic surgery.

A tissue defect that can be treated with the compositions and methods as described herein can be any injury resulting from damage of tissue at a tissue defect site or at the interface of the muscle and skeleton, which can be due to strenuous activity.

Methods, systems, and compositions described herein can provide support for surgical and tissue defect/injury sites in tissue-tissue interfaces and multi-tissue interfaces, including but not limited to ligament/tendon-to-bone insertion, articular cartilage-to-bone junction, hip labrum, intervertebral disc (nucleus pulposus-annulus fibrosus-endplates), cementumperiodontal ligament-alveolar bone, muscle-to-tendon, inhomogeneous or anisotropic tissues such as knee meniscus or temporomandibular joint disc, root-periodontium complex, tendon-bone insertion, synovial joints, or fibrocartilaginous tissues.

Repair of the tissue defects listed above, especially tendon-to-bone repair, presents a challenging mechanical problem: repairs require high strength and resilience to accommodate forces from activities of daily living and to avoid repair site elongation or rupture; however, the bond between a compliant tendon and stiff bone causes high stress concentrations that limit attachment strength.

The healthy tendon enthesis facilitates load transfer from tendon to bone in several ways, including by (i) distributing force over a relatively large footprint area to reduce local stresses, (ii) using a compliant transitional fibrocartilaginous tissue to optimize stress concentrations and toughen the attachment, and (iii) interdigitating fibrocartilage with bone. Unfortunately, current surgical repair techniques fail to recreate these stress-dissipation mechanisms, leading to high failure rates.

Rotator cuff injury is one example of a tendon-to-bone injury that is notoriously challenging to repair. Post-repair rupture rates range from 20% for young, healthy athletes with small tears to as high as 94% for massive tears in elderly patients. These failure rates are not surprising from a mechanical perspective: single- and double-row rotator cuff repairs transfer almost all of the force from muscle to bone across two anchor points, where the suture from a bone anchor punctures through the tendon. The vast majority (86%) of rotator cuff repair ruptures occur by the tendon pulling through the sutures at those anchor points. Due to these high failure rates, operative rotator cuff repair is only indicated for a subset of patients with symptomatic (i.e., painful) shoulders. In other words, repairs can be performed to reduce pain without reinstating shoulder function. Approximately half of the US population over 60 years old has a rotator cuff tear, leading to over 500,000 repairs of symptomatic shoulders annually. Approximately $500 million per year is spent on repairs that rupture. With a growing aging and elderly population, improving on these failure rates is critical to reinstate shoulder function in these patients.

While there have been many improvements in surgical techniques for rotator cuff repair over the last several decades, the current standard of care using sutures and anchors can be a crude mechanical solution. The current standard of care for repairing a torn rotator cuff consists of grasping the tendon with a suture and securing it to the bone using anchors. This results in load transfer from tendon to bone across the few anchor points and a high risk of failure at the tendon-suture or tendon-anchor interface.

Furthermore, current care does not include any biologic augmentation at the repair site, and the typically poor healing at the tendon-bone interface is insufficient to mechanically integrate the tendon to the bone. Several biologic grafts have been tested clinically for enhancing rotator cuff repair, but none have shown efficacy. Conventional grafts have been unsuccessful primarily due to their use as patches over the repair site (i.e., there are not interposed between the healing tendon and bone) and their poor mechanical properties.

As another example, there are approximately 10,000 flexor tendon repairs annually (USA). Furthermore, the elongation rate is 48% and the rupture rate is 6-10%. Most failures happen within the first 6 weeks.

The two primary reasons for failed repair are (1) poor initial mechanical fixation of the tendon and bone and (2) a lack of regeneration of the native tendon-to-bone attachment structure. Similarly, re-attachment is a clinical challenge in other load-bearing tissues as well, including anterior cruciate ligament reconstruction, meniscus repair, and Achilles tendon repair.

To address these issues, the present disclosure provides an adhesive film that can deliver cells or growth factors to the repair. The adhesive properties increase the initial attachment strength of the tendon-to-bone repair by reducing local elevations of stress, and the cells and growth factors can enhance the biology of tendon-to-bone healing. While the experiments described in the present disclosure are focused on the rotator cuff, it is understood that the disclosure can be readily applied to other anatomical sites in orthopedics (e.g., anterior cruciate ligament reconstruction, meniscus repair, Achilles tendon repair) as well as other surgical specialties (e.g., hernia repair, skin closure).

As mentioned above, 20-94% of rotator cuff repairs fail, primarily due to poor fixation of the tendon to the bone. The adhesive films disclosed herein can reduce this failure rate, leading to significantly fewer revision surgeries by applying fixation across the entire tendon-to-bone footprint area. The adhesive films disclosed herein result in improved mechanical fixation of tendon to bone, delivery of bioactive factors for enhanced healing, and delivery of cells for enhanced healing.

Generally, the critical period for tendon repair is in the first 6 weeks. An adequate mechanical solution needs to hold the tendon together for long enough (about 6 weeks) for a body to heal sufficiently. A suture or suture anchor system can be a crude mechanical solution that does not use the length of the suture to transfer load most effectively.

### Connective Tissue Growth Factor and Cells

The materials and methods as described herein can comprise a connective tissue growth factor. For example, the growth factor can be CTGF/CCN2.

A growth factor (e.g., CTGF/CCN2) can direct fibroblast differentiation from human mesenchymal stem/stromal cells. A growth factor can be associated with wound healing and fibrosis or heparin binding for sustained release.

CTGF/CCN2 is available from a variety of commercial sources. Preferably, the connective tissue growth factor is a human connective tissue growth factor.

Other growth factors that can be used in the methods and compositions as described herein can be a growth factor selected from CTGF, TGFβs (e.g., TGFβ3), CTGF, BMPs (e.g., BMP12), SDF, bFGF, IGF, GDF, PDGF (e.g., PDGF-BB), VEGF, or EGF or their isoforms.

A growth factor can be supplied at, for example, a concentration of about 0 to about 1000 ng/mL. For example, CTGF can be present at a concentration of about 10, about 20, about 30, about 40, about 50, about 60, about 70, about 80, about 90, about 100, about 110, about 120, about 130, about 140, about 150, about 160, about 170, about 180, about 190, about 200, about 250, about 300, about 350, about 400, about 450, about 500, about 550, about 600, about 700, about 750, about 800, about 850, about 900, about 950, or about 1000 ng/ml. For example, CTGF can be present at a concentration of about 100 ng/ml.

A polymer or hydrogel can be used as a structure to deliver adhesive, growth factor, or cells.

Delivery methods for growth factors include, but are not limited to, hydrogel-based delivery systems, microsphere-based delivery systems, and fibrin-based delivery systems. Cells can be seeded directly onto the films for delivery.

As an example, the polymeric delivery system can be a polymeric microsphere, e.g., a PLGA polymeric microspheres. A variety of polymeric delivery systems, as well as methods for encapsulating a molecule such as a growth factor, are known to the art (see e.g., Varde and Pack 2004 Expert Opin Biol Ther 4, 35-51). Polymeric microspheres can be produced using naturally occurring or synthetic polymers and are particulate systems in the size range of 0.1 μm to 500 μm. Polymeric micelles and polymeromes are polymeric delivery vehicles with similar characteristics to microspheres and can also facilitate encapsulation and matrix integration of the compounds described herein. Fabrication, encapsulation, and stabilization of microspheres for a variety of payloads are within the skill of the art (see e.g., Varde & Pack (2004) Expert Opin. Biol. 4 (1) 35-51). The release rate of the microspheres can be tailored by type of polymer, polymer molecular weight, copolymer composition, excipients added to the microsphere formulation, and microsphere size. Polymer materials useful for forming microspheres include PLA, PLGA, PLGA coated with DPPC, DPPC, DSPC, EVAc, gelatin, albumin, chitosan, dextran, DL-PLG, SDLMs, PEG (e.g., ProMaxx), sodium hyaluronate, diketopiperazine derivatives (e.g., Technosphere), calcium phosphate-PEG particles, and/or oligosaccharide derivative DPPG (e.g., Solidose). Encapsulation can be accomplished, for example, using a water/oil single emulsion method, a water-oil-water double emulsion method, or lyophilization. Several commercial encapsulation technologies are available (e.g., ProLease®, Alkerme). The selection of an encapsulation agent can depend on the film or adhesive selected.

As described herein, cells can be delivered to the tissue. A cell can be a living cell, a population of cells, a mesenchymal stromal cell, a tendon-derived stem cell, or an adipose-derived progenitor. The cell can be seeded directly onto the film or the layer. The hydrogel or polymer is a delivery agent for the cell. The hydrogel or polymer is interspersed with the cells. The cell can be delivered by a cell sheet within an adhesive film or layer.

### Patterning

As described herein, spatial patterns and combinations of adhesive agents, growth factors, and/or other materials including hydrogels and water can be designed to provide optimized strength, toughness, stiffness, and stress redistribution. One embodiment involves adhesives staggered with non-adhesive agents to arrest propagating cracks within the adhesive layer. Another embodiment involves adhesives randomly interspersed with a non-adhesive hydrogel to reduce the stiffness of the adhesive layer. Another embodiment involves patterning with a non-adhesive hydrogel to create a gradient in adhesion strength along the length of the film. Another embodiment involves adhesives with variable molecular length interspersed to promote adhesion across length scales. Another embodiment involves patterning in the thickness of adhesives.

### Parameters

As described herein, material (e.g., adhesive) or repair parameters can be optimized according to specific tissue needs.

In some embodiments, the shear modulus (Ga) value of a material or repair can be between about 10³ and about 10¹⁰ Pa. For example, the shear modulus value can be about 10³ Pa, about 10⁴ Pa, about 10⁵ Pa, about 10⁶ Pa, about 10⁷ Pa, about 10⁸ Pa, about 10⁹ Pa, or about 10¹⁰ Pa. Recitation of each of these discrete values is understood to include ranges between each value. Recitation of each of a range is understood to include discrete values within the range.

In some embodiments, the failure shear stress (T_{fail}) value of a material or repair is between about 10⁵ Pa and about 10⁷ Pa. For example, the failure shear stress (T_{fail}) value can be about 10⁵ Pa, about 10⁶ Pa, or about 10⁷ Pa. Recitation of each of these discrete values is understood to include ranges between each value. Recitation of each of a range is understood to include discrete values within the range.

In some embodiments, the expected strength improvement from an adhesive film or adhesive layer, calculated based on material and geometric properties of the adhesive and repair, can be between about 3% and about 3000% of the current strength of repairs without adhesive films or adhesive layers. For example, the expected repair strength improvement value can be about 0%; about 100%; about 200%; about 300%; about 400%; 500%; about 600%; about 700%; about 800%; about 900%; about 1000%; about 1100%; about 1200%; about 1300%; about 1400%; about 1500%; about 1600%; about 1700%; about 1800%; about 1900%; about 2000%; about 2100%; about 2200%; about 2300%; about 2400%; about 2500%; about 2600%; about 2700%; about 2800%; about 2900%; or about 3000% of the strength of repairs without adhesive films or adhesive layers. Recitation of each of these discrete values is understood to include ranges between each value. Recitation of each of a range is understood to include discrete values within the range.

In some embodiments, the strength (maximum load (N)) value of a material or repair can be between about 0 N and about 3000 N. For example, the strength (maximum load (N)) value of a material or repair can be about 0 N; about 100 N; about 200 N; about 300 N; about 400 N; 500 N; about 600 N; about 700 N; about 800 N; about 900 N; about 1000 N; about 1100 N; about 1200 N; about 1300 N; about 1400 N; about 1500 N; about 1600 N; about 1700 N; about 1800 N; about 1900 N; about 2000 N; about 2100 N; about 2200 N; about 2300 N; about 2400 N; about 2500 N; about 2600 N; about 2700 N; about 2800 N; about 2900 N; or about 3000 N. Recitation of each of a range is understood to include discrete values within the range. Recitation of each of a range is understood to include discrete values within the range.

In some embodiments, the strength, the toughness, the resilience, or the stiffness of the adhesive or repair will be optimized for the specific damaged tissue.

### Formulation

As described herein, an adhesive or agents can be made into a film and/or applied to a film and implanted into a subject, directly applied into a subject, or applied to a material such as a suture before being implanted into a subject. Such materials, adhesives, or agents can be a formulated adhesive or agent. Also described herein, a material, scaffold, or agent of the present disclosure can be implanted in a subject. Such a material, scaffold, or agent can include various pharmaceutically acceptable carriers or excipients.

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc., may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the present disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of treating a musculoskeletal injury in a subject in need of administration of a therapeutically effective film, so as to improve a surgical outcome.

Methods described herein are generally performed on a subject in need thereof. A subject in need of the therapeutic methods described herein can be a subject having, diagnosed with, suspected of having, or at risk for developing a musculoskeletal injury. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans. For example, the subject can be a human subject.

Generally, a safe and effective amount of film or agent is, for example, that amount that would cause the desired therapeutic effect in a subject while minimizing undesired side effects. In various embodiments, an effective amount of film or agent described herein can substantially inhibit risk for post-surgical injury, slow the progress of post-surgical injury, or limit the development of post-surgical injury.

According to the methods described herein, administration of the film to the subject can be of any surgical method known in the art, such as implantation.

When used in the treatments described herein, a therapeutically effective amount of adhesive or agent (e.g., growth factor) can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to result in an improved surgical outcome.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Treatment in accord with the methods described herein can be performed prior to, concurrent with, or after conventional treatment modalities for a musculoskeletal injury.

An adhesive or agent can be administered to the film or subject simultaneously or sequentially with another agent. An agent or composition can be administered in combination with a film as described herein wherein the film can be a biodegradable, biocompatible polymeric film that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in contact with or in proximity to a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10:0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to a material or agent, such as a film, a layer, or an adhesive. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, or sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3 d ed., Cold Spring Harbor Laboratory Press, ISBN-10:0879695773; Elhai, J. and Wolk, C. P. 1988. Methods in Enzymology 167, 747-754; Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Mechanically Competent Chitosan-Based Bioadhesive for Tendon-to-Bone Repair

This example describes an underwater-setting, mussel-inspired, catechol-derived bioadhesive that is not only biocompatible, but also improves tendon-to-bone healing both biologically and mechanically. It is superior in both regards to commercially available tissue adhesives.

Current suture-based surgical techniques used to repair torn rotator cuff tendons do not result in mechanically competent tendon-to-bone attachments, leading to high postoperative failure rates. Although adhesives have been proposed to protect against sutures tearing through tendon during healing, no currently available adhesive meets the clinical needs of adhesive strength, biocompatibility, and promotion of healing. Here, a biocompatible, graded, 3,4-dihydroxy phenyl chitosan (BGC) bioadhesive designed to meet these needs is presented. Although 3,4-dihydroxy phenyl chitosan (DP-chitosan) bioadhesives are biocompatible, their adhesion strength is low; soluble oxidants or cross-linking agents can be added for higher bonding strength, but this sacrifices biocompatibility. These challenges are overcome by developing a periodate-modified ion exchange resin-bead filtration system that oxidizes catechol moieties to quinones and filters off the activating agent and resin. The resulting BGC bioadhesive exhibited sixfold higher strength compared to commercially available tissue adhesives, with strength in the range necessary to improve tendon-to-bone repair (≈1 MPa, ≈20% of current suture repair strength). The bioadhesive is biocompatible and promoted tenogenesis; cells exposed to the bioadhesive demonstrated enhanced expression of collagen I and the tenogenic marker Scx. Results demonstrated that the bioadhesive has the potential to improve the strength of a tendon-to-bone repair and promote healing.

### Introduction

Surgical repair of connective tissues to bone is a major clinical challenge. Stress concentrations arise at the interface of these load-bearing tissues because of differences in their mechanical properties (e.g., the modulus of tendon is two orders of magnitude smaller that the modulus of bone). Nature's solution to this mechanical challenge is to generate a functionally graded transition at the interface, with spatial gradients in structure and composition, to facilitate load transfer and avoid stress concentrations. Unfortunately, despite substantial efforts to develop mechanical and biologic therapies to recreate this attachment system after injury, conventional approaches have largely failed. For example, surgical repair of rotator cuff tendons to the humeral head relies on a finite number of suture grasping points (typically 2 or 4). Load transfer is therefore concentrated on these grasping points, resulting in stress concentrations and clinical failure rates of 20% in young healthy patients with small tears and 94% in elderly patients with massive tears. This unsolved clinical problem will continue to worsen with an aging population and associated rotator cuff tears requiring surgical repair.

A bioadhesive capable of reconnecting tendon and bone could change the paradigm of conventional repair techniques. Rather than concentrating load transfer across a small number of suture anchor points, a bioadhesive with appropriate mechanical properties may be able to spread load over a larger area, reduce stress, and eventually entirely replace surgical sutures. Significant effort has been devoted to the development of bioadhesives, with the desired features of biocompatibility, biodegradability, adhesive strength, and fast curing on wet surfaces. However, most approaches either lack the strength necessary for orthopedic applications (e.g., fibrin “glue”) or have a poor biocompatibility profile (e.g., cyanoacrylates). Efforts to mimic marine organisms, which form strong attachments in aqueous environments (e.g., mussels, barnacles), have not yielded bioadhesives that meet the design criteria necessary to improve tendon-to-bone repair, generally failing by an order of magnitude to provide appropriate adhesive strength and toughness.

In exemplary embodiments of the present disclosure, a biocompatible, graded, 3,4-dihydroxy phenyl chitosan (BGC) bioadhesive was developed and composed in two forms: i) a rigid, dried dihydroxy phenyl chitosan (DP-chitosan) sheet which adhered well to bone surfaces and ii) a viscous DP-chitosan gel which adhered well to tendon surfaces. The strong oxidizing agent sodium periodate was used to accelerate curing and enhance the adhesive strength of the DP-chitosan by rapid oxidation of catecholic (3,4-dihydroxyl phenyl) moieties to quinones, which covalently cross-link other catechols, phenols, and amine residues that are present. Periodate oxidizes catecholin a stoichiometric −2H+ and −2e− oxidation to o-quinone. Therefore, it has previously been used to achieve higher wet-bonding strength by triggering oxidative cross-linking. However, periodate is a very strong oxidizing agent, and its toxicity has limited its use in biological applications. To overcome this, periodate anions were immobilized onto cationic resin beads and developed a technique to filter these off of the adhesive. The result was rapid delivery of quinone-rich adhesive to the tendon, with excess periodate (IO4−) and iodate (IO3−) anions removed with the filtered resin beads. Lap shear testing demonstrated that the BGC bioadhesive had substantially greater adhesive strength than commercially available adhesives, and that its mechanical properties approached the range required for enhancing tendon-to-bone repair. Furthermore, in vitro studies demonstrated that the bioadhesive was biocompatible and promoted tenogenesis.

### Results

### Design of a BGC Bioadhesive

Chitosan was chosen as the backbone for the bioadhesive because it is biocompatible and biodegradable, with low immunogenicity, high osteoconductivity, effective antimicrobial behavior, and favorable wound-healing properties. DP-chitosan adhesives, inspired by marine mussel catechol chemistry, are soluble in neutral or low acidity aqueous media, and have demonstrated potential for non-load-bearing biomedical applications. However, these adhesives are weak and require toxic oxidizing agents like sodium periodate or cross-linkers to achieve shear bonding strength and modulus relevant to orthopedic applications.

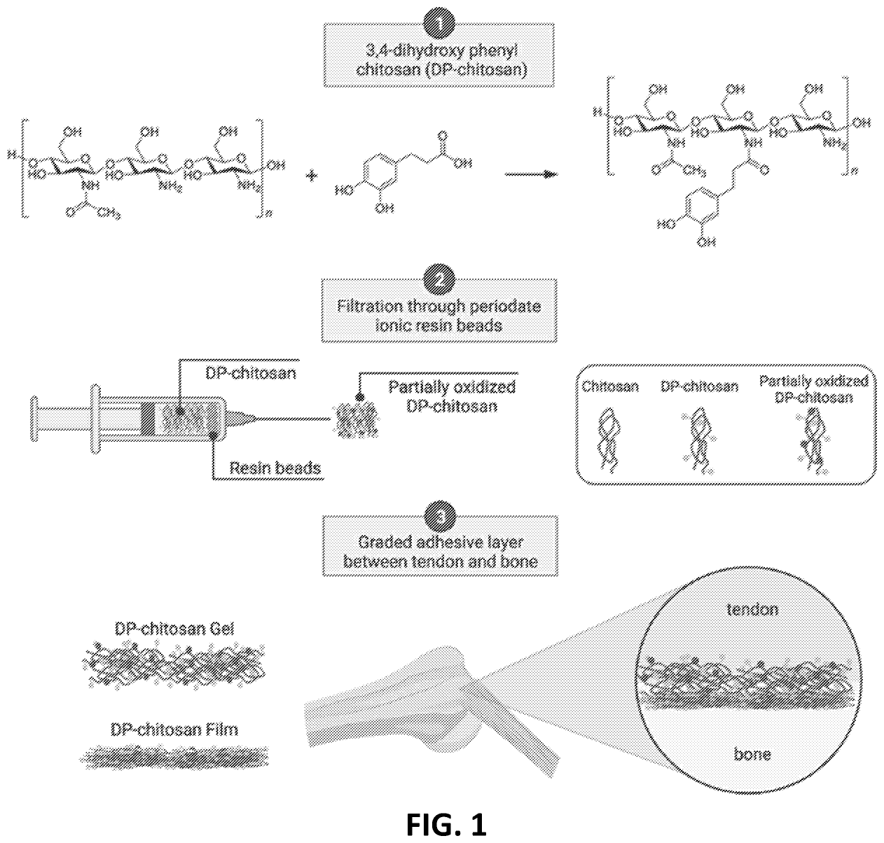

To address the issue of toxicity, a biocompatible viscous DP-chitosan was synthesized by immobilizing sodium periodate onto ion exchange resin beads, mixing them with DP-chitosan, and filtering the mixture to remove the toxic chemicals after oxidation (FIG. 1). In this approach, a rapid gelation (curing) occurred in approximately 30 s; DP-chitosan was mixed with the oxidizing agent-immobilized resin beads, filtered, and applied in less than 30 s on wet tissue surfaces.

Additionally, a mechanical compatibility issue had to be overcome because of the vastly different surface chemistry and material properties of tendon and bone. Due to the sensitivity of interfacial stress concentrations caused by material property mismatches, a single formulation was not effective for adhesion to both tendon and bone. Preliminary lap shear tests using the viscous gel showed excellent adherence to tendon but poor adherence to bone. In contrast, a thin sheet form of BGC showed excellent adherence to bone but poor adherence to tendon. Therefore, a biomimetic graded adhesive was constructed to mimic the graded nature of the native attachment. To achieve a gradient in adhesive density, BGC bioadhesive was prepared in two forms: 1) a viscous gel, formed after filtering DP-chitosan solution/colloid and 2) a thin sheet, formed after drying DP-chitosan solution/colloid. The dried DP-chitosan sheet was applied onto the bone surface, the viscous partially oxidized DP-chitosan gel was placed onto the tendon surface, and the two were clamped together. In this manner, a graded bioadhesive layer formed between tendon and bone and cured over time.

### Mechanical Properties of the BGC Bioadhesive

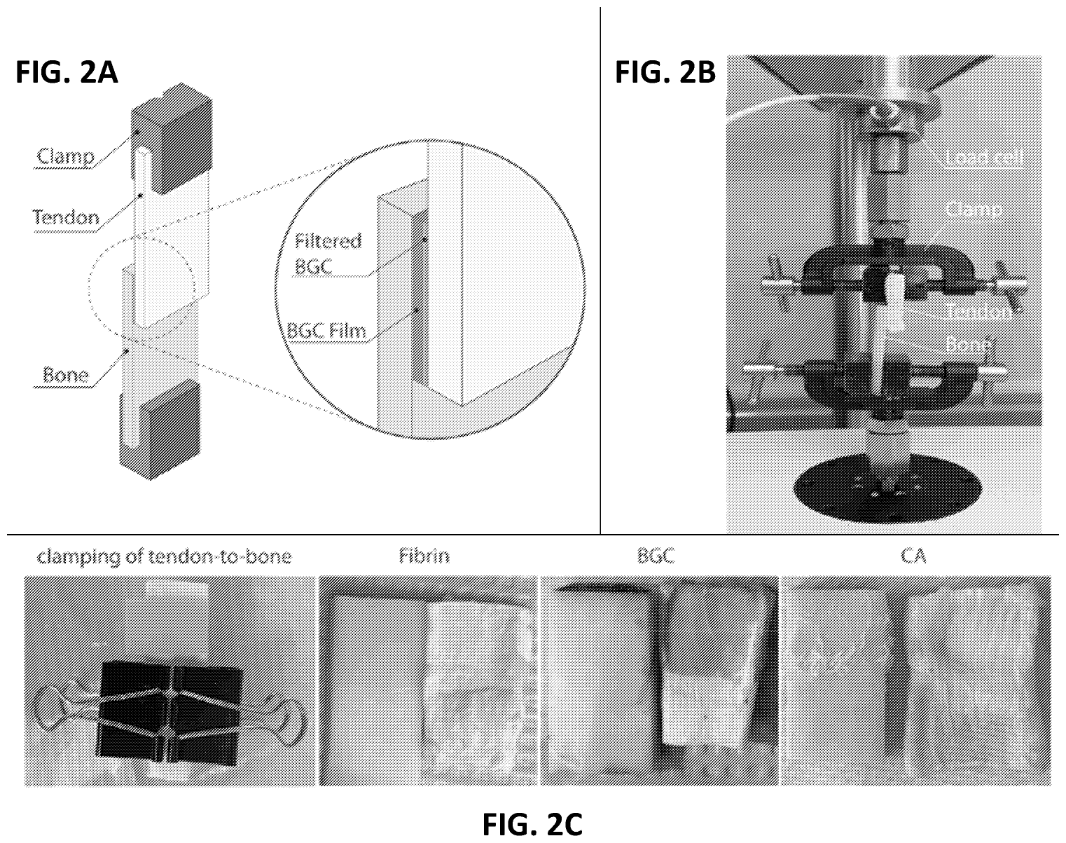

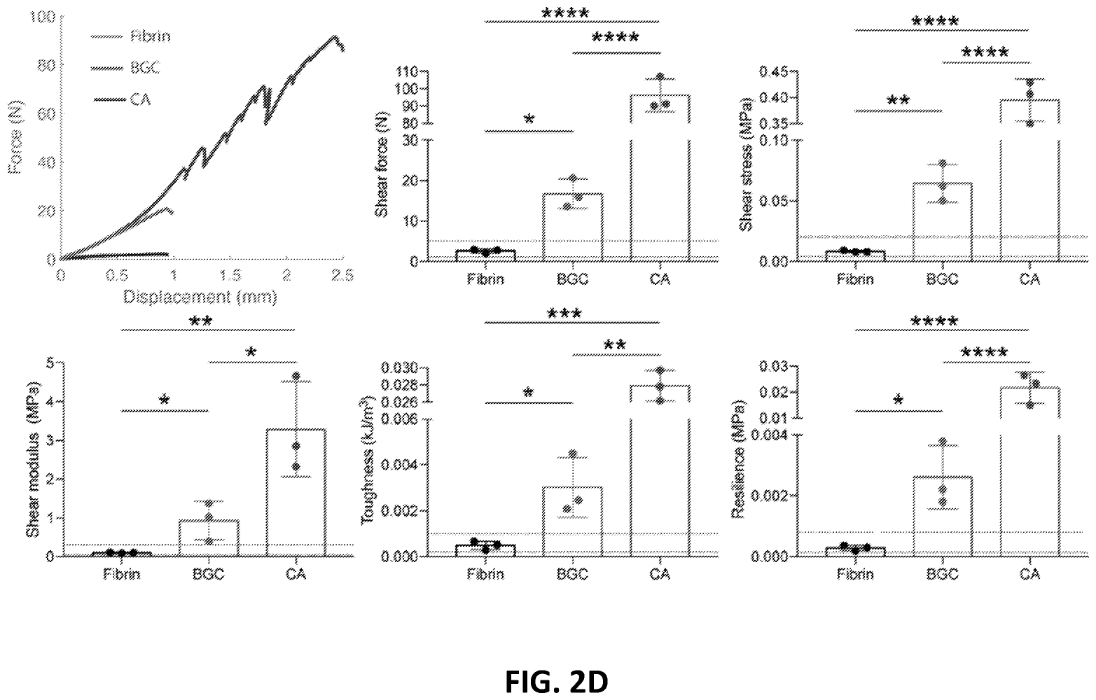

To investigate the mechanical function of the BGC bioadhesive, lap shear tests were performed for tendon planks adhered to bone planks. The BGC bioadhesive was compared to fibrin, a clinically approved adhesive for use inside the body, and to cyanoacrylate (CA), a clinically approved adhesive for use outside the body (e.g., for skin lacerations). Fibrin, which forms rapidly via reaction of fibrinogen and thrombin, has excellent biocompatibility and is currently used for treating postoperative bleeding. However, typical formulations of fibrin do not come close to the adhesive strength necessary for adhering musculoskeletal tissues together. CAs, on the other hand, demonstrate rapid curing and very high adhesive strength, but are highly cytotoxic, preventing their use for orthopaedic applications. Tendon and bone planks were adhered with either fibrin, BGC bioadhesive, or CA and then clamped overnight (FIG. 2A). The tendon-bone assembly was then pulled to failure in uniaxial tension in a lap shear configuration (FIG. 2B). In the fibrin-adhered samples, failure was apparent within the fibrin layer (FIG. 1C). In contrast, intact BGC bioadhesive was observed only the surface of tendon after failure, indicating that the weak link in the sample was BGC adhesion to bone. In CA-adhered samples, failure was observed in the tendon, with ruptured collagen fibers remaining on the bone surface. Although the BGC bioadhesive had lower adhesive strength compared to CA, it was 3-4 times stronger than the strongest bioadhesive reported in the literature (dotted horizontal lines in FIG. 1D). Furthermore, it was approximately eightfold stronger (i.e., higher shear force, shear stress, FIG. 1D), ninefold stiffer (i.e., shear modulus), and six-fold tougher (i.e., toughness) than fibrin.

### Biocompatibility of the BGC Bioadhesive

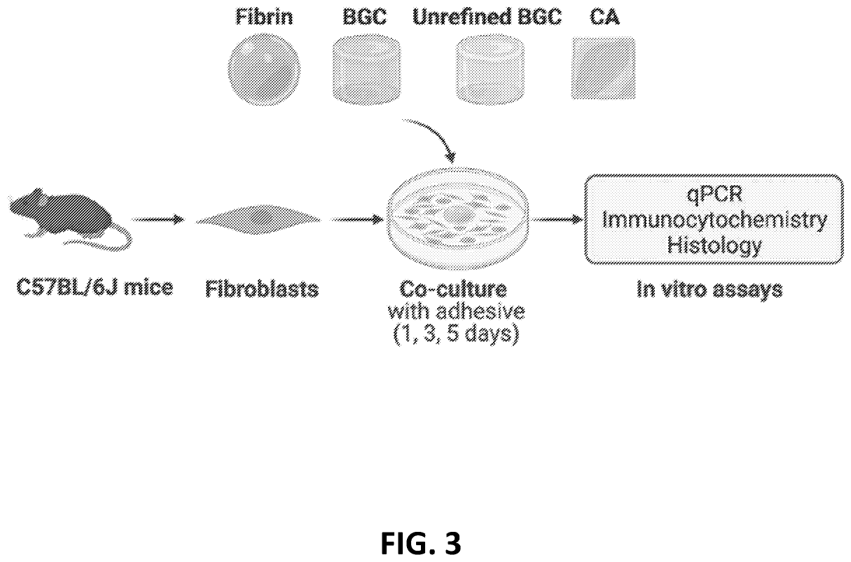

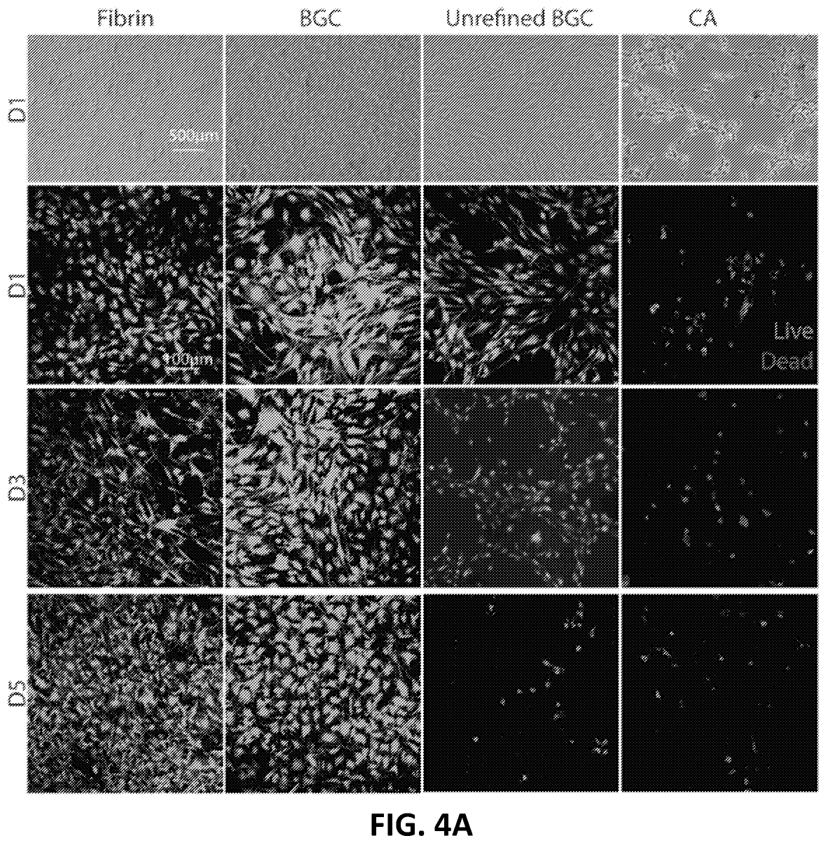

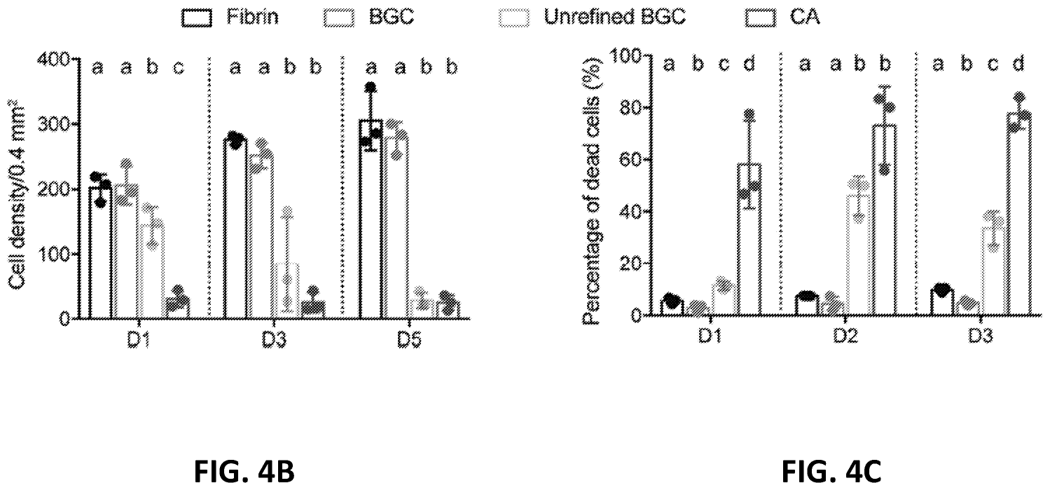

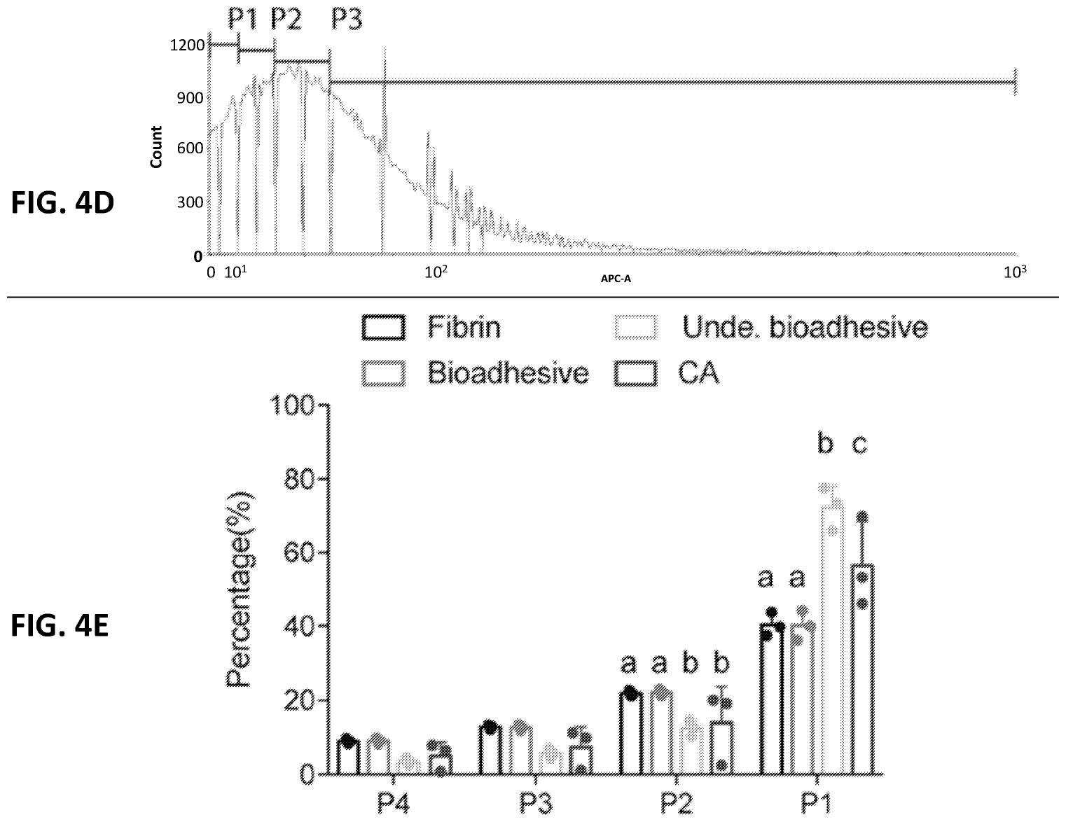

The effects of the adhesives on cell viability and bioactivity were evaluated by culturing primary fibroblasts from C57BL/6J mouse tail tendons in the presence of the adhesives (FIG. 3). Cultures that included CA showed few viable cells, illustrating high cytotoxicity (FIG. 4A). In cultures that included unrefined BGC (i.e., BGC that was not filtered through the anion exchange beads to remove periodate), some spindle-shaped cells were observed, with marginally higher numbers of cells compared to CA at day 5. In contrast to CA and unrefined BGC, both fibrin and BGC groups showed significantly higher numbers and density of cells, with cells having spindle shaped morphologies (FIG. 4B). Cell density decreased significantly between day 1 and day 5 when culturing tendon fibroblasts with unrefined BGC, but did not decrease in the BGC group, demonstrating a clear benefit of the novel filtration system used to remove the toxic chemical after oxidation. Live-dead staining revealed significantly more dead cells in the undefined BGC and CA groups than in the fibrin and BGC groups (FIG. 4C). Furthermore, significantly fewer dead cells were found in BGC cultures compared to fibrin cultures.

### Promotion of Tenogenesis by the BGC Bioadhesive

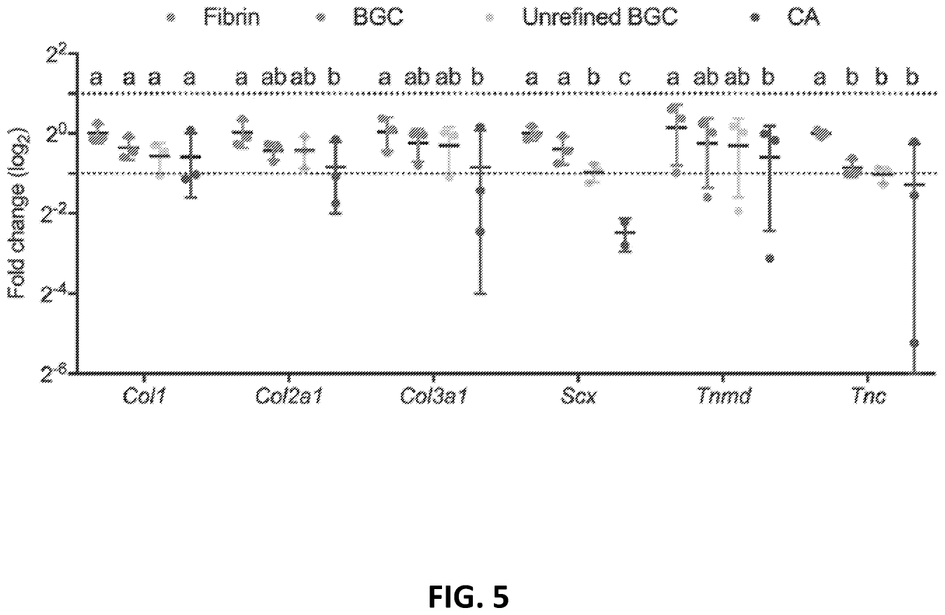

In addition to the requirements that the adhesive is biocompatible, cures on wet surfaces, and has appropriate mechanical properties, an ideal bioadhesive promotes tenogenesis and synthesis of extracellular matrix proteins at the healing interface. To evaluate the capacity of the BGC bioadhesive to promote tenogenesis, gene expression of tenogenic markers (i.e., Col1a1, Col2a1, Col3a1, Scx, Tnmd, Tnc) and protein production of the tenogenic transcription factor Scx and the primary extracellular matrix component collagen type I were evaluated after culturing primary tendon-derived cells with the different adhesives for 1 day (FIG. 5).

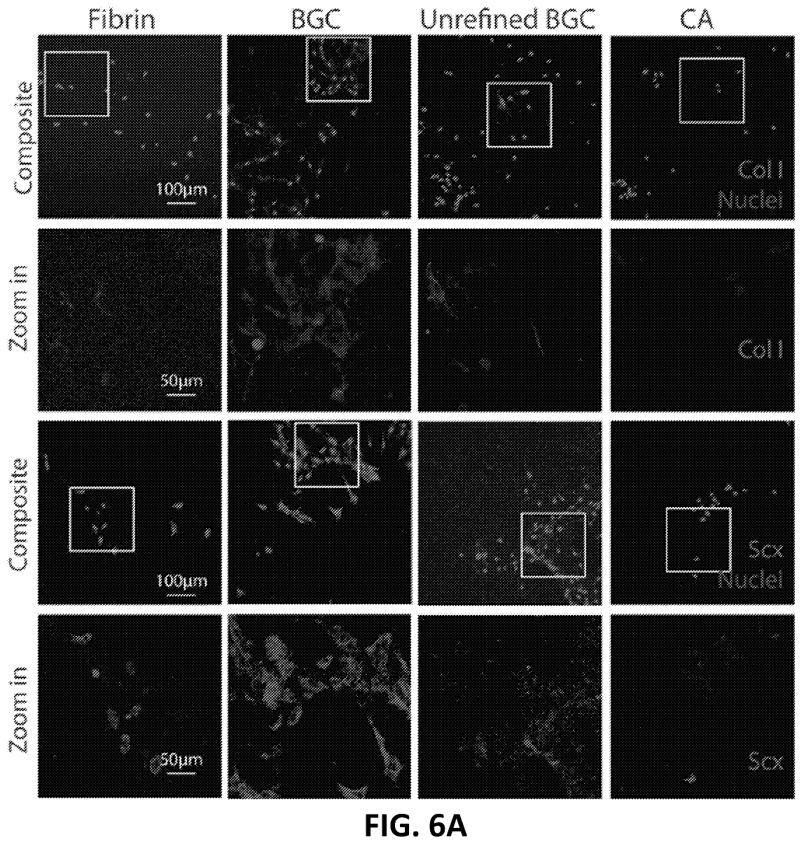

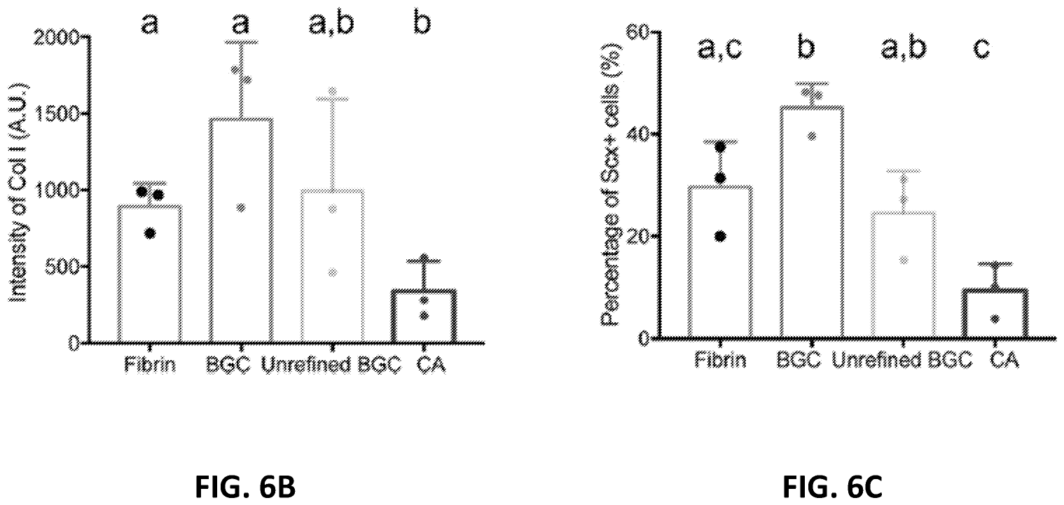

Compared to unrefined BGC and CA, the BGC bioadhesive induced higher expression of Scx. There were no significant differences in the expression of Col1a1, Col2a1, Col3a1, and Tnmd. However, these results must be interpreted in the context of the toxic environments of unrefined BGC and CA, where there were substantially fewer viable cells compared to fibrin and BGC. qPCR analysis was performed only on cells that survived, and total RNA expression was substantially lower in the unrefined BGC and CA groups than in the fibrin and BGC groups. To account for this issue, immunocytochemistry was conducted to evaluate protein expression in cells cultured with the different adhesives. Cells cultured on the BGC bioadhesive exhibited the highest deposition of collagen type I and Scx (FIG. 6A). Collagen type I expression was significantly higher for cells cultured with BGC bioadhesive than those cultured with CA (FIG. 6B). Significantly more cells expressed Scx when cultured with BGC than those cultured with fibrin or CA (FIG. 6C).

### Discussion

The BGC bioadhesive developed here maintained cell viability, promoted tenogenesis, and achieved adhesion strength 3-4 times higher than that of previously published protein bioadhesives. The exceptional biocompatibility of the BGC bioadhesive was realized through a novel filtration system, which removed toxic oxidizing reagents while facilitating crosslinking of the bioadhesive. Although the precise amount of toxic periodate sequestered by the bead filtration system was not quantified, in vitro results demonstrated that the system was effective in preventing any biologically meaningful cytotoxic effects. This approach uniquely improved adhesive strength without sacrificing biocompatibility. The BGC bioadhesive was designed and evaluated for tendon-to-bone repair applications, demonstrating clinically relevant adhesive strength and tenogenic potential.

Previous studies have reported that chitosan exhibits adhesive strength superior to that of fibrin due to its ability to bind to exposed amino groups in applications such as healing of bone fracture. Furthermore, chitosan has been demonstrated to have osteo-conductive and inductive properties, e.g., serving as a supplement to bone grafts for large bone defects and as a bioadhesive for small, fragmented bone fractures. Chitosan-based scaffolds/matrices also promoted chondrogenic and/or osteogenic differentiation of mesenchymal stem cells, depending on the local environment near the implant, and maintained the phenotype of mature chondrocytes. As described herein, tenogenic potential of a chitosan-based adhesive was observed. The potential of chitosan to promote tenogenesis, chondrogenesis, and osteogenesis makes it a promising candidate for promoting the regeneration of the tendon-to-bone attachment, which requires simultaneous formation of tendon, fibrocartilage, and bone. However, the molecular mechanisms underlying the regulation of chitosan on tenogenesis, chondrogenesis, and osteogenesis are unclear; further in vitro and in vivo work is needed to define how chitosan affects stem cell differentiation and mature cell responses. Due to the presence of functional groups such as catecholic moieties and amines, chitosan can also be chemically and physically modified to acquire new or enhanced biophysical and biochemical properties (e.g., oxidized, as discussed herein). Therefore, the adhesive and biological properties of chitosan-based bioadhesives may be leveraged simultaneously to prevent rupture post repair and to promote tendon-to-bone healing in the long term.

A key obstacle to the use of bioadhesives in tendon-to-bone repair is their low adhesive strength compared to physiological levels of force. The present disclosure addresses this using a graded BGC bioadhesive approach, with a stiff BGC film adhered to the stiff bone surface and a soft BGC gel adhered to the soft tendon surface. During the curing process, these layers integrated to form a functionally graded bioadhesive between the tendon and bone. In some embodiments, the BGC bioadhesive achieved the strength necessary to augment existing approaches in tendon-to-bone repair. Importantly, the adhesive may be used in conjunction with conventional surgical repair techniques, with the surgeon applying the adhesive between tendon and bone as a treatment adjuvant to suturing. Implementation of a mixing syringe for dispensing the bioadhesive may add negligible time to the procedure, with gelation and oxidation occurring within minutes after dispensing. As conventional repair approaches result in compression of sutured tendon onto the bone, excellent adhesion would be expected during the early period after repair while the adhesive is curing. In some embodiments, the BGC bioadhesive shows potential for repair of cartilage, ligament, meniscus, and intervertebral disc, and for functioning as a carrier for delivering growth factors and pharmaceutical reagents.

### Experimental Section

### Biocompatible, Graded, DP-Chitosan Bioadhesive Synthesis—General Information

All synthetic manipulations were conducted in a fume hood. Catechol-functionalized Chitosan (DP-chitosan) was synthesized by coupling chitosan (low molecular weight, Aldrich cat. #448869) and 3-(3,4-dihydroxyphenyl) propionic acid (98%, Alfa cat. Aesar #A11638) using 1-ethyl-3-(3-dimethylaminopropyl) carbodiimide hydrochloride (EDC·HCl, Chem Impex) as an activating agent, N-hydroxysuccinimide (e.g., NHS, Acros) as a coactivator, and 2,6-lutidine (Acros) as a base. The pH was monitored using indicator strips (VWR, intermediate range) with a resolution of 0.5 pH units. 200 proof ethanol (EtOH, molecular biology grade, denatured with 5% isopropanol) and deionized water were used as reaction media. Dowex 1×8 (50-100 mesh, beads, Chloride form) was obtained from BeanTown Chemical, and sodium periodate was obtained from Fisher. All other solvents were ACS grade and were obtained from Fisher and used as received.

### Biocompatible, Graded, DP-Chitosan Bioadhesive Synthesis—Synthesis of DP-Chitosan Polymers

DP-chitosan was prepared by reacting Solution A with Solution B as follows. Solution A: 9.5 mL of conc. aq. HCl was added to 290 mL DI water in a 500 mL round bottom flask equipped with a large ellipsoidal magnetic stir bar. Stirring was performed at ≈800 rpm. 4 g of powdered chitosan (equivalent to ≈30 mmol of glucosamine monomer) was then added gradually, with vigorous stirring until fully dissolved to obtain a soluble solution of chitosan. Sufficient lutidine was added in 0.5 mL portions with stirring and pH monitoring to achieve a pH of 5.0. Solutions B1-B4: A separate solution was prepared by dissolving 6.285 g of 3-(3,4-dihydroxyphenyl) propionic acid (34.5 mmol) and 4.367 g of NHS (37.95 mmol) in 200 mL of EtOH. After complete dissolution, the solution was divided into four separate vials (labeled B1, B2, B3, and B4 respectively) such that the volumes were nearly identical. These vials were capped until further use.

Coupling of the catecholic acid to chitosan was achieved through addition of 1.776 g of EDC·HCl to solution B1 to activate the carboxylic acid. The vial was re-capped and shaken until the EDC had completely dissolved and shaking was continued for an additional 2-3 min. After mixing, the entire contents of B1 were added to the stirred solution A. The pH of the reaction was continuously monitored and lutidine was added in 0.25 mL portions to maintain a pH of 5.0. Additional EtOH and DI water were added as necessary to maintain a homogeneous reaction medium if viscosity was observed to increase to a point where mixing was compromised.

30 min after the addition of B1 to solution A, the above procedure was repeated. 1.776 g EDC·HCl was added to vial B2. The entire volume of B2 was added to solution A, with pH monitored and adjusted as necessary. This process was repeated at 30 min intervals until all “B” solutions had been added and a stable pH of 5.0 was maintained for at least 2 h after the final addition of B4. The reaction vessel was sealed with a fresh rubber septum, purged with Argon, and stirred for an additional 18 h at ambient temperature.

### Biocompatible, Graded, DP-Chitosan Bioadhesive Synthesis—Workup, Isolation, and Purification of DP-Chitosan

The reaction mixture was poured slowly into 2 L of acetone in a 3 L Erlenmeyer flask with gradual swirling, causing a mass of material to precipitate. The mixture was stirred briefly and allowed to stand, and the supernatant was decanted off. 2 L of EtOH was then added, the flask was swirled/agitated for 10 min and allowed to stand, and the supernatant was decanted off. The solid residue was then extracted with 1 L of CH2Cl2, in which it was swirled for 10 min and allowed to settle, after which the CH2Cl2 was decanted off; this process was performed three times. Residual CH2Cl2 was removed by trituration 3× with methanol. Wet material was then transferred to a round bottom flask, and remaining volatiles were removed under high vacuum until a constant weight was achieved. The amount of DP-chitosan yielded by this method was 4.7 g of a light brown, hygroscopic powder.

### Biocompatible, Graded, DP-Chitosan Bioadhesive Synthesis—Reaction Notes

Reaction pH was maintained at 5.0 to prevent catechol auto-oxidation and subsequent cross-linking. If reaction pH remained above 5.5 for extended periods of time, oxidative cross-linking was rapid, yielding a dark brown material which was substantially more difficult to dissolve. Reaction pH below 4.5 resulted in a very slow rate of coupling, and in significantly fewer catechol moieties being incorporated into the chitosan polymer.

### Biocompatible, Graded, DP-Chitosan Bioadhesive Synthesis—Oxidatively Cross-linked DP-Chitosan

(DP-Chitosan 5.5): To obtain a slightly oxidatively cross-linked form of DP-chitosan (termed DP-chitosan 5.5, to differentiate it from the other material), the above procedure was followed, except with a pH of 5.0-5.5, rather than 4.5-5.0, during the reaction. Yield under these conditions was similar, giving a darker brown material more resistant to dissolution. This material was used to prepare the DP-chitosan sheet described below.

### Biocompatible, Graded, DP-Chitosan Bioadhesive Synthesis—DP-Chitosan Sheet

Slightly oxidatively cross-linked DP-chitosan 5.5 was pulverized into a fine powder, then dissolved in dilute aqueous HCl (pH=2-3) by stirring/ultrasonication at a concentration of 1% wt./v. (10 mg mL⁻¹). 20 mL of this solution was poured into a 140 mm diameter plastic petri dish that was placed in an incubator. Water was evaporated to form a thin dry sheet by passing dry air over the dish at 55° C. overnight. After the dish was removed from the incubator and allowed to cool, the sheet was gently peeled from the dish with tweezers and stored in a plastic bag until further use.

### Biocompatible, Graded, DP-Chitosan Bioadhesive Synthesis—DP-Chitosan Adhesive Solutions

To prepare DP-chitosan adhesive solutions, DP-chitosan was pulverized into a fine powder with a mortar and pestle, weighed, and added slowly to a vigorously stirred dilute solution of aqueous HCl (pH=2-3). After ≈30 min, the solution was subjected to ultrasonication in an ultrasonic cleaning bath, and alternately ultrasonicated/stirred at ambient temperature until a clear solution was obtained, with no visible undissolved material. Solutions up to ≈6.25% wt./v. may be obtained readily by this method. Concentrated colloids of DP-chitosan (>6.25% wt./v.) were obtained by using a pulse ultra-sonic homogenizer (Fisher). Sonication was continued at 25 kHz, 55 W, pulse on/off at 5/20 s until powder was dissolved.

### Biocompatible, Graded, DP-Chitosan Bioadhesive Synthesis—Polymer Resin-Supported Periodate

To prepare the periodate anion exchange resin, 25 g of Dowex 1×8 and 20 g of sodium periodate (Fisher) were mixed and stirred gently in 200 ml of distilled water for 6 h. The supernatant was decanted off and the resin was treated with a fresh solution of 20 g sodium periodate in 200 mL distilled water, stirring again gently for additional 6 h. The resin beads were then filtered off and washed successively on a Buchner funnel with 200 ml distilled water four times, then twice with 100 mL of tetrahydrofuran, and then twice with 100 mL of di-ethyl ether, then transferred to a round bottom flask and dried overnight under high vacuum. The dry resin was transferred to a vial, tightly sealed, and stored in the dark at 4° C. Because periodate is a strong oxidizing agent, aqueous washings containing periodate were segregated from organic waste, handled, and disposed of with care, and excess periodate in the combined supernatant/aqueous washings was decomposed by slow addition of a concentrated solution of table sugar.

### Biocompatible, Graded, DP-Chitosan Bioadhesive Synthesis—Preparation of Resin Syringes and Method of Dispensation

A 3 mL disposable luer lock syringe was fitted with a 10 μm PTFE membrane filter at the outlet, and the plunger was removed to facilitate addition of periodate resin. Periodate resin beads were weighed and added to the syringe. For each 1.5 mL of 6.25% wt./wt. DP-chitosan solution, resin loading was tested at 12.7, 38.1, 57.1, and 76.2 mg, corresponding to resin/DP-chitosan mass ratios of 13.5%, 40.6%, 53.6%, and 81.2%, respectively. For each 1.5 mL of 10% wt./wt. DP-chitosan solution, resin loading was tested at 20.3, 61.4, and 92.1 mg (13.5, 40.6, 53.6% wt./wt. resin/DP-chitosan, respectively). Before dispensation, the syringe was agitated to allow the beads to settle at the bottom of the chamber. To dispense, the syringe was held vertically, and 1.5 mL of DP-chitosan solution was added quickly to the periodate-resin syringe, whereupon the plunger was reinserted and rapidly (t<30 s) pressed down to pass the solution through the resin and filter, concomitantly oxidizing and dispensing DP-chitosan while allowing the resin beads to remain behind within the syringe. Higher resin loadings required rapid dispensation to avoid crosslinking/setting of the adhesive within the syringe. Preliminary lap shear mechanical tests were conducted to determine the optimal amount of resin beads to oxidize DP-chitosan. These pilot tests indicated that a 2 mL 10% wt./wt. DP-chitosan solution with 50 mg resin beads achieved the highest adhesive strength.

### Lap Shear Mechanical Testing

A series of tendon and bone planks were prepared from fresh frozen bovine deep digital flexor tendons and femurs, respectively. Bovine flexor tendons were cut into planks and the top and bottom of these tendons were leveled using a cryostat into planks of ≈20 mm width, 35 mm length, and 10 mm height. Bovine femurs were sawed and the top and bottom surfaces were finished into smooth planks of ≈20 mm width, 40 mm length, and 10 mm height. Three groups (n=3/group) of tendon specimens were adhered to bone planks using fibrin, BGC bioadhesive, or CA, and mechanically tested in a lap shear configuration. For the fibrin group, 20 mg mL⁻¹ fibrinogen (F3879, MilliporeSigma) mixed with 100 units mL⁻¹ thrombin (10 602 400 001, MilliporeSigma) was gelatinized for 10 min and evenly coated onto the tendon and bone surfaces. The tendon-to-bone planks were clamped using two clips (FIG. 1A). For the BGC bioadhesive group, DP-chitosan colloid was filtered through periodate-resin beads in a syringe connected to a 0.2 μm filter and evenly spread over the tendon surface. A dried DP-chitosan sheet was placed onto the bone surface wetted by PBS (phosphate-buffered saline) and tendon and bone were clamped as mentioned above. For the CA group, CA was spread over the tendon and bone surfaces prior to clamping. All clamped tendon-bone planks were kept in PBS overnight at 5° C. to allow for complete adhesive curing. After five cycles of sinusoidal-ramp pre-conditioning to 0.1 mm displacement, tendon-bone planks were pulled in uniaxial tension at 0.3 mm s⁻¹ until failure using a material testing machine (ElectroForce 3200, TA instruments). Mechanical parameters were calculated using recorded force, time, and displacement, as described previously.

### Fibroblast Harvest and Culture

Tail tendons from 8-week-old C57BL/6J mice (n=3/group) were dissected and digested in medium containing αMEM medium, 4 mg mL⁻¹ collagenase type 2, 5% fetal bovine serum, and 1% penicillin/streptomycin for 2 h at 37° C. The digestion solution was filtered through a 40 μm strainer and cultured in growth medium containing αMEM medium, 10% fetal bovine serum, and 1% penicillin/streptomycin until 80-90% confluency for the downstream experiment.

### Gene Expression Evaluation

0.5×10⁶ cells were seeded on the 6-well plates and cultured in the growth medium overnight. The cells were starved in medium containing αMEM medium, 0.5% fetal bovine serum, and 1% penicillin/streptomycin for 6 h. The starved cells were cocultured with similar amounts of fibrin, BGC, unrefined BGC, and CA in the growth medium for 18 h. The cocultured cells were digested and mRNA was extracted using RNeasy Mini Kit (Qiagen) according to the manufacturer's protocol. The purified RNA was reverse transcribed using a high-capacity cDNA reverse transcription kit (Invitrogen). The relative abundance of tendon maker genes was examined by SYBR Green-based quantitative reverse transcriptional polymerase chain reaction (RT-PCR) on an Applied Biosystems Quant-Studio 6 flex system. Glyceraldehyde 3-phosphate dehydrogenase was selected as housekeeping gene and the relative mRNA expression of each marker gene was presented as 2−ΔΔCt.

### Cell Viability and Immunocytochemistry Analysis

As described above, cells on sterile glass coverslips were cocultured with fibrin, BGC, unrefined BGC, and CA for 18 h, 3 d, or 5 d and used for cell viability and immunocytochemistry analysis. A LIVE/DEAD Viability kit (L3224, ThermoFisher) was used for cell viability evaluation. Briefly, the cells were washed with PBS after the removal of the growth medium and stained with 1×10⁻⁶ m calcein-AM and 2×10⁻⁶ m ethidium homodimer-1 for 45 min at room temperature. The coverslips with cells were then mounted and sealed on glass slides for imaging. For immunocytochemistry analysis, cells were fixed in 4% paraformaldehyde for 10 min, washed in PBS, and then incubated in 0.5% Triton X-100/PBS for 10 min. After three washes, cells were blocked in 15% goat serum/PBS, incubated in Collagen I antibody (NB600, Novus Biologicals) or Anti-Scx antibody (ab58655, Abcam), and an appropriate secondary antibody for 1 h. The cover slips were then mounted on slides with mounting media and imaged on a Nikon Ti Eclipse inverted microscope. Cell density was calculated as the cell number per 0.4 mm2. The percentage of dead cells was counted as the number of dead cells normalized to the total number of cells. The intensity of collagen type I was determined by normalizing its intensity over the region of interest. The percentage of Scx+ cells was determined as the number of cells with Scx expression normalized to the total number of cells.

### Statistical Analysis

All data were presented as mean±standard deviation of three biological replicates for lap shear testing and for in vitro assays. Statistical analysis was conducted in GraphPad Prism 7. Data points (and hence sample sizes) were shown within each bar. Data were not preprocessed. All parameters were compared by one-way ANOVA followed by Tukey's post hoc tests when appropriate.

What is claimed is:

1. A biocompatible adhesive for use in a subject having a tissue defect, wherein the biocompatible adhesive comprises an oxidized or crosslinked chitosan and optionally does not comprise an oxidizing agent or crosslinking agent.

2. A biocompatible film comprising the biocompatible adhesive of claim 1 for use in a subject having a tissue defect, wherein the biocompatible adhesive comprises an oxidized chitosan or crosslinked chitosan.

3. A biocompatible material comprising the biocompatible adhesive of claim 1 for use in a subject having a tissue defect, wherein the biocompatible adhesive and the biocompatible film comprises an oxidized or crosslinked chitosan.

4. A method of repairing a tissue defect at a tissue defect site comprising:
(i) providing the biocompatible film of claim 2;
(ii) contacting the biocompatible film with the biocompatible adhesive;
(iii) applying the biocompatible film to a tissue defect (e.g., bone); and
(iv) applying the biocompatible adhesive to the biocompatible film or a tissue defect (e.g., tendon).

5. A method of repairing a tissue defect at a tissue defect site comprising:
(i) applying a primer to one side of the tissue defect; and
(ii) applying the biocompatible adhesive of claim 1 to the primed tissue defect.

6. The method of claim 5, in which the primer is amino-bisphosphonate or a mixture of amino-phosphonates.

7. A method of generating a biocompatible film, biocompatible adhesive, or biocompatible material comprising:
removing an oxidizer in an oxidized chitosan, resulting in a substantially oxidizer-free oxidized chitosan biocompatible adhesive; and
optionally drying the biocompatible adhesive resulting in a biocompatible film or applying the biocompatible adhesive to the biocompatible film.

8. The biocompatible adhesive of claim 1, wherein the oxidizing agent or the crosslinking agent has been removed by filtration.

9. The biocompatible adhesive of claim 1, wherein the chitosan is HP-chitosan and is made by reacting primary amine groups of chitosan with carboxylic acid groups of dihydroxy or trihydroxybenzene-(CH₂)n-COOH, n=0-10.

10. The biocompatible adhesive of claim 9, wherein gelation of the HP-chitosan is achieved using a filterable oxidizing crosslinking agent.

11. The biocompatible adhesive of claim 10, wherein the filterable oxidizing crosslinking agent is a periodate form of ionic exchange resins.

12. The biocompatible adhesive of claim 1, wherein the biocompatible adhesive is filtered to remove and/or recycle the oxidizing agent, wherein the oxidizing agent is immobilized onto resin beads.

13. The biocompatible adhesive of claim 12, wherein the oxidizing agent is immobilized onto a porous material, foam, fibrous material, or coated porous, foam, or fibrous material.

14. The biocompatible adhesive of claim 1, wherein a periodate-modified ion exchange resin-bead filtration system is used to oxidize catechol moieties.

15. The biocompatible adhesive of claim 14, wherein the catechol moieties are oxidized to quinones.

16. The biocompatible adhesive of claim 12, wherein the filtration filters off an activating agent and resin.

17. The biocompatible film of claim 2,
comprising di- or tri-hydroxy phenyl-functionalized chitosan (HP-chitosan).

18. The biocompatible film of claim 2, wherein:
the biocompatible film has a shear modulus value between about 1 MPa and about 5 MPa.

19. (canceled)

20. The biocompatible film of claim 2, wherein:
the biocompatible film has a shear stress value between about 0.1 MPa and about 0.45 MPa.

21. (canceled)

22. The biocompatible film of claim 2, wherein:
the biocompatible film comprises a rigid, dried HP-Chitosan sheet.

23-47. (canceled)
